(19) United States
(12) Patent Application Publication
(10) Pub. No.: US 2026/0056185 A1
(43) Pub. Date: Feb. 26, 2026

(54) COMPOSITIONS AND METHODS FOR TREATING AND DETECTING CANCER

(71) Applicant: Case Western Reserve University, Cleveland, OH (US)

(72) Inventors: Susann Brady-Kalnay, Wellington, OH (US); Kathleen Molyneaux, Cleveland Heights, OH (US)

(21) Appl. No.: 19/245,853

(22) Filed: Jun. 23, 2025

Related U.S. Application Data
(63) Continuation-in-part of application No. PCT/US2023/085889, filed on Dec. 26, 2023.
(60) Provisional application No. 63/477,025, filed on Dec. 23, 2022.
(60) Provisional application No. 63/520,443, filed on Aug. 18, 2023.

(51) Int. Cl. G01N 33/50; G01N 33/574

(52) U.S. Cl. CPC G01N 33/5011; G01N 33/5091; G01N 33/57492

(57) ABSTRACT

A pharmaceutical composition comprising a compound that targets and/or binds to a binding pocket at interface between the D1 and D2 domains of an intracellular portion or fragment of the receptor protein tyrosine phosphatase (RPTP) IIb cell adhesion molecules (e.g., PTPμ) and that is capable of inhibiting RPTP mediated adhesion of cells and/or cancer cell growth and/or sphere formation.

### RELATED APPLICATION

This application is a Continuation-in-Part of PCT/US2023/085889, filed Dec. 26, 2023, which claims priority from U.S. Provisional Application No. 63/477,025, filed Dec. 23, 2022 and 63/520,443, filed Aug. 18, 2023, the subject matter of which are incorporated herein by reference in their entirety.

### SEQUENCE LISTING

The instant application contains a Sequence Listing which has been submitted electronically in XML format and is hereby incorporated by reference in its entirety. Said XML copy, created on Dec. 21, 2023, is named CWR-032372WO ORD.st.26 and is 2,507 bytes in size.

### BACKGROUND

Hyperactivation of protein phosphorylation is a hallmark of many cancers and has been shown to drive survival, growth and metastasis. This has stimulated the development of therapeutic agents designed to inhibit the catalytic activity of kinases, enzymes that phosphorylate specific substrates typically on serine, threonine and/or tyrosine residues. Modulation of phosphatases, which dephosphorylate downstream targets, also results in disease. However, attempts to target protein tyrosine phosphatases (PTPs) have been considerably less successful than those targeting kinases. Drugs able to interact with the highly charged active sites of tyrosine phosphatases are often not membrane permeable. Also, the active site within the phosphatase domains is highly conserved at the amino acid level meaning targeting agents are likely to be promiscuous. Thus, the phosphatase field has turned its focus towards developing allosteric inhibitors. These agents have the potential to be both more membrane permeable and specific because they may interact with regulatory regions that could be both less charged and more divergent than the catalytic domain.

The protein tyrosine phosphatase mu (PTPμ) is an attractive therapeutic target because its expression is altered in many cancers, with consequent changes in growth and cell motility. PTPμ is known to stimulate neuronal or axonal migration (neurite outgrowth) during development of the nervous system. The expression of full-length PTPμ inhibits cell growth and motility and induces contact inhibition thus functioning as a tumor suppressor. Proteolytic cleavage of PTPμ reverses this effect and generates both extracellular and intracellular fragments that actually stimulate migration of glioblastoma cells and thus function as oncogenes. There are structural motifs within both the extracellular and intracellular fragments that can be targeted for drug development.

Allosteric regions of the intracellular domain of PTPμ can also be exploited for drug development. The intracellular portion of PTPμ consists of a juxtamembrane region resembling the cytoplasmic region of cadherins and two highly conserved phosphatase domains, a catalytically active membrane proximal D1 domain and a D2 phosphatase domain. Agents targeting a known regulatory motif termed the ‘wedge’ domain within the juxtamembrane portion of receptor protein tyrosine phosphatase (RPTPs) show therapeutic potential, although the precise role of the wedge domain remains controversial. Peptides targeting this region in LAR and PTPσ have been shown to affect downstream signaling, and the PTPσ wedge peptide (intracellular sigma peptide) is being developed as a therapeutic agent for promoting neural regeneration after injury. Peptides derived from the sequence of the PTPμ wedge domain as well as compounds targeting a wedge-adjacent pocket have been shown to inhibit the migration of glioma cells in culture. Notably, a compound targeting a wedge-adjacent pocket was shown to inhibit PTPμ phosphatase activity in vitro.

Like the wedge domains, the D2 domains of RPTPs have controversial functions, but these too have begun to garner interest as potential sites for drug development driven by the recent proliferation of complete crystal structures for RPTP intracellular domains. Historic studies have indicated that the D2 domain may promote or inhibit the catalytic activity of the D1 domain, may affect substrate specificity, control protein dimerization or intra-molecular interactions. It may also have phosphatase or other enzymatic activities, such as a denitrase in some contexts. As of yet, there is no crystal structure encompassing both the D1 and D2 domains of PTPμ to aid drug development.

### SUMMARY

Embodiments described herein relate to compounds that target receptor protein tyrosine phosphatase (RPTP) cell adhesion molecules (e.g., PTPμ), and particularly a binding pocket at an interface between the D1 and D2 domains of an intracellular portion or fragment of an RPTP, and that are capable of inhibiting RPTP mediated adhesion of cells and/or cancer cell growth and/or sphere formation and/or phosphatase activity as well as to their use in methods of treating cancer in a subject in need thereof and methods of detecting, monitoring, and/or imaging cancer cells and/or cancer cell metastasis, migration, dispersal, and/or invasion in a subject.

In some embodiments, the compound can have the structure of formula (I):

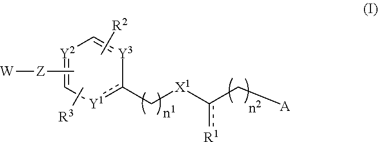

or a pharmaceutically acceptable salt, tautomer, or solvate thereof,
wherein:
a dashed line (e.g., ---- or --) is an optional bond;
A is cycloalkyl, aryl, heteroaryl, or heterocyclyl, each of which is optionally substituted with one or more R⁴;
X¹ is CH₂ or O;
Y¹, Y², and Y³ are each independently C(H)ₘ or N(H)ₙ;
W is absent or an aryl or heteroaryl, each optionally substituted with one or more R^{3a};
Z is absent or an alkylene, -alkylene-cycloalkylene-, -alkylene-heterocyclylene-, or —N(R⁶)—, each optionally substituted with one or more R⁵;
R¹ and R⁵ are each independently O, —N(R⁶)₂, halogen, hydroxyl, alkyl, haloalkyl, or alkoxy;
R², R³, and each R^{3a} are each independently absent, —N(R⁶)₂, halogen, hydroxyl, alkyl, haloalkyl, alkoxy;
R⁴ is ═O, halogen, hydroxyl, alkyl, alkoxy, haloalkyl, aryl, or haloaryl;
R⁶ is H, halogen, hydroxyl, alkyl, alkoxy, or haloalkyl;
m is 0, 1, or 2;
n and n¹ are each independently 0 or 1; and
n² is 0, 1, 2, or 3.

In some embodiments, A is a dihydro-indenyl, indenyl, indolinyl, indolyl, isoindolyl, indolizinyl, indazolyl, benzimidazolyl, azaindolyl, pyrazolo-pyrimidinyl, purinyl, benzofuranyl, isobenzofuranyl, or benzothiophenyl, each of which is optionally substituted with one or more R⁴.

In other embodiments A is selected from:

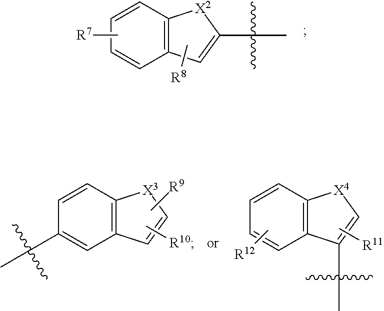

wherein X², X³, and X⁴ are each independently N(R¹³) or CH₂,
R⁷, R⁸, R⁹, and R¹⁰ are each independently absent, halogen, hydroxyl, alkyl, alkoxy, or haloalkyl;
R¹¹ and R¹² are each independently absent, halogen, hydroxyl, alkyl, alkoxy, haloalkyl, aryl or haloaryl; and
R¹³ is H, halogen, hydroxyl, alkyl, alkoxy, or haloalkyl.

In some embodiments, X² is N(R¹³) and R⁷ and R⁸ are each independently absent, halogen, hydroxyl, C₁-C₆ alkyl, C₁-C₆ alkoxy, or C₁-C₆ haloalkyl.

In some embodiments, X³ is N(R¹³) and R⁹ and R¹⁰ are each independently absent, halogen, hydroxyl, C₁-C₆ alkyl, C₁-C₆ alkoxy, or C₁-C₆ haloalkyl.

In some embodiments, X⁴ is N(R¹³) and R¹¹ is absent, aryl or haloaryl and R¹² is absent, a halogen, or C₁-C₆ alkyl.

In some embodiments, A is selected from

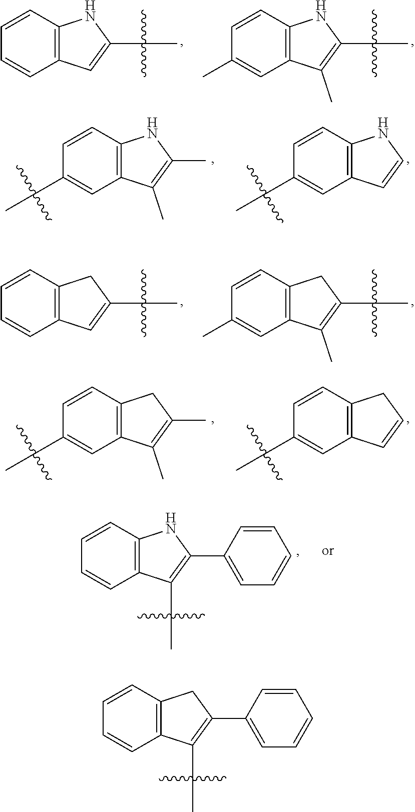

each of which is optionally substituted with one or more halogen.

In some embodiments, R¹ is O.

In some embodiments, X¹ is O.

In some embodiments, n¹ is 1; n² is 0; Y¹, Y², and Y³ are each independently N; R² is —N(R⁶)₂, preferably, —NH₂; and R³ is absent.

In some embodiments, n¹ is 0; n² is 0; Y¹, Y², and Y³ are each independently CH; and R² and R³ are absent.

In some embodiments, Z is:

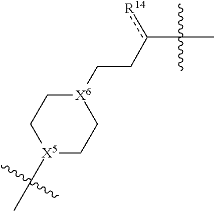

or —N(H)—; X⁵ and X⁶ are each independently CH or N; R¹⁴ is O, —N(H)₂, halogen, hydroxyl, alkyl, haloalkyl, or alkoxy; and W is an aryl or heteroaryl, each optionally substituted with one or more R^{3a}.

In other embodiments, Z is

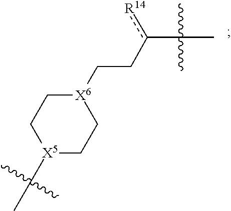

X⁵ and X⁶ are each independently CH or N, preferably N; R¹⁴ is O; W is an aryl; and R^{3a} is absent, halogen, or alkoxy.

In some embodiments, Z is —N(H)—; W is an aryl; and R^{3a} is absent.

In some embodiments, n¹ is 1; n² is 1, 2 or 3; Y¹, Y², and Y³ are each independently N; W and Z are absent; and R² and R³ can each independently be —N(R⁶)₂, preferably R⁶ is H.

In some embodiments, A is

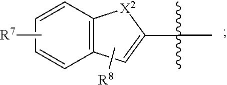

n¹ is 0;
n² is 0;
X¹ is 0;
X² is N(R¹³);
Y¹, Y², and Y³ are each independently C(H);
Z is

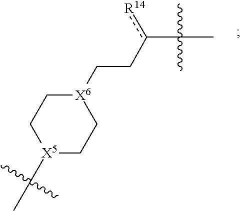

W is an aryl optionally substituted with one or more R^{3a};
X⁵ and X⁶ are each independently N;
R¹ is O;
R² and R³ are absent;
each R^{3a} is absent, halogen, alkyl, or alkoxy;
R⁷ and R⁸ are each independently absent, halogen, hydroxyl, alkyl, alkoxy, or haloalkyl, preferably an alkyl, and more preferably a C₁-C₆ alkyl;
R¹³ is H, halogen, hydroxyl, alkyl, alkoxy, or haloalkyl; and
R¹⁴ is O, —N(H)₂, halogen, hydroxyl, alkyl, haloalkyl, or alkoxy, preferably, O.

In other embodiments,
A is

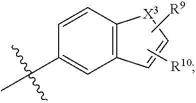

n¹ is 1;
n² is 0;
X¹ is O;
X³ is N(R¹³);
Y¹, Y², and Y³ are each independently N;
Z is —N(H)—;
W is an aryl optionally substituted with one or more R^{3a};
R¹ is O;
R² and R³ are each independently absent, —N(R⁶)₂, halogen, hydroxyl, alkyl, haloalkyl, alkoxy;
R^{3a} is absent, halogen, alkyl, or alkoxy;
R⁶ is H, halogen, hydroxyl, alkyl, alkoxy, or haloalkyl;
R⁹ and R¹⁰ are each independently absent, halogen, hydroxyl, alkyl, alkoxy, or haloalkyl, preferably an alkyl, and more preferably a C₁-C₆ alkyl; and
R¹³ is H, halogen, hydroxyl, alkyl, alkoxy, or haloalkyl.

In still other embodiments,
A is

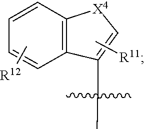

n¹ is 1;
n² is 1, 2, or 3;
X¹ is O;
X⁴ is N(R¹³);
Y¹, Y², and Y³ are each independently N;
Z and W are absent;
R¹ is O;
R² and R³ are each independently absent, —N(R⁶)₂, halogen, hydroxyl, alkyl, haloalkyl, alkoxy;
R⁶ is H, halogen, hydroxyl, alkyl, alkoxy, or haloalkyl;
R¹¹ and R¹² are each independently absent, halogen, hydroxyl, alkyl, alkoxy, haloalkyl, aryl or haloaryl, and preferably R¹² is a halogen and R¹¹ is an aryl or haloaryl; and
R¹³ is H, halogen, hydroxyl, alkyl, alkoxy, or haloalkyl.

Other embodiments described herein relate to pharmaceutical composition that includes a compound of formula (IA):

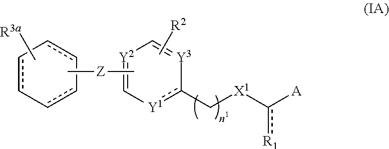

or a pharmaceutically acceptable salt, tautomer, or solvate thereof,
wherein:
a dashed line (e.g., ---- or --) is an optional bond;
A is cycloalkyl, aryl, heteroaryl, or heterocyclyl, each of which is optionally substituted with one or more R⁴;
X¹ is CH₂ or O;
Y¹, Y², and Y³ are each independently C(H)ₘ or N(H)ₙ;
Z is an alkylene, -alkylene-cycloalkylene-, -alkylene-heterocyclylene-, or —N(R⁶)—, each optionally substituted with one or more R⁵,
R¹ and R⁵ are each independently O, —N(R⁶)₂, halogen, hydroxyl, alkyl, haloalkyl, or alkoxy;
R² and each R^{3a} are each independently absent, —N(R⁶)₂, halogen, hydroxyl, alkyl, haloalkyl, alkoxy;
R⁴ is ═O, halogen, hydroxyl, alkyl, alkoxy, or haloalkyl;
R⁶ is H, halogen, hydroxyl, alkyl, alkoxy, or haloalkyl;
m is 0, 1, or 2; and
n and n¹ are each independently 0 or 1.

In some embodiments, A is a dihydro-indenyl, indenyl, indolinyl, indolyl, isoindolyl, indolizinyl, indazolyl, benzimidazolyl, azaindolyl, pyrazolo-pyrimidinyl, purinyl, benzofuranyl, isobenzofuranyl, benzothiophenyl, each of which is optionally substituted with one or more R⁴.

In some embodiments, A is

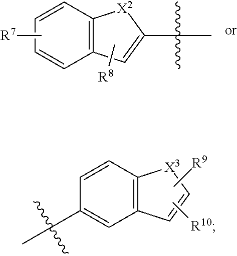

X² and X³ are each independently N(R¹³) or CH₂; R⁷, R⁸, R⁹, and R¹⁰ are each independently absent, halogen, hydroxyl, alkyl, alkoxy, or haloalkyl; and R¹³ is H, halogen, hydroxyl, alkyl, alkoxy, or haloalkyl.

In some embodiments, X² is N(R¹³).

In some embodiments, X³ is N(R¹³).

In other embodiments, A is selected from

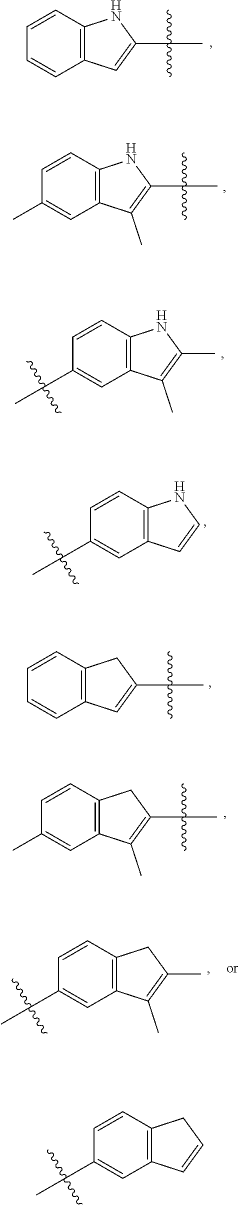

each of which is optionally substituted with one or more halogen.

In some embodiments, R¹ is O.

In some embodiments, X¹ is O.

In some embodiments, n¹ is 1 and Y¹, Y², and Y³ are each independently N. R² is —N(R⁶)₂, preferably, —NH₂.

In some embodiments, n¹ is 0 and Y¹, Y², and Y³ are each independently CH. R² is absent.

In some embodiments, Z is:

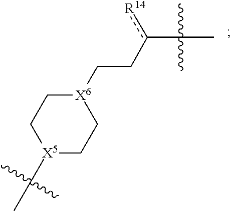

or —N(H)—; X⁵ and X⁶ are each independently CH or N; and R¹⁴ is O, —N(H)₂, halogen, hydroxyl, alkyl, haloalkyl, or alkoxy.

In still other embodiments, Z is —N(H)—, and R^{3a} is absent.

Still other embodiments relate to a pharmaceutical composition that includes a compound of formula (II):

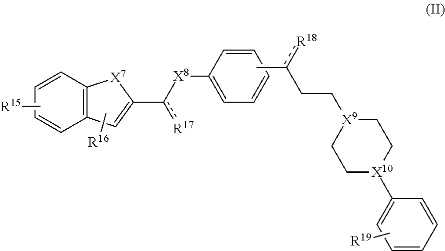

or a pharmaceutically acceptable salt, tautomer, or solvate thereof;
wherein,
a dashed line (e.g., ---- or --) is an optional bond;
X⁷ is N(R²⁰) or CH₂;
X⁸ is CH₂ or O;
X⁹ and X¹⁰ are each independently CH or N;
R¹⁵ and R¹⁶ are each independently absent, halogen, hydroxyl, alkyl, alkoxy, or haloalkyl;
R¹⁷ and R¹⁸ are each independently O, —N(R²⁰)₂, halogen, hydroxyl, alkyl, haloalkyl, or alkoxy;
R¹⁹ is absent, —N(R²⁰)₂, halogen, hydroxyl, alkyl, haloalkyl, alkoxy; and
each R²⁰ is independently H, halogen, hydroxyl, alkyl, alkoxy, or haloalkyl.

In some embodiments, X⁷ is N(R²⁰); X⁸ is O; X⁹ and X¹⁰ are N; R¹⁵ and R¹⁶ are each independently absent, halogen, hydroxyl, C₁-C₆ alkyl, C₁-C₆ alkoxy, or C₁-C₆ haloalkyl; R¹⁷ and R¹⁸ are each independently O; R¹⁹ is absent, halogen, or alkoxy; and R²⁰ is H, halogen, C1-C₆ alkyl, or C₁-C₆ haloalkyl.

Still other embodiments relate to a pharmaceutical composition that includes a compound of formula (III):

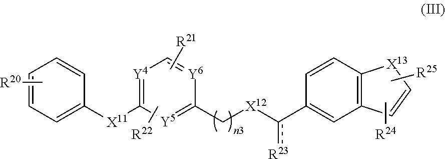

or a pharmaceutically acceptable salt, tautomer, or solvate thereof;
wherein:
a dashed line (e.g., ---- or --) is an optional bond;
X¹¹ and X¹³ are each independently N(R²⁶) or CH₂;
X¹² is CH₂ or O;
Y⁴, Y⁵, and Y⁶ are each independently C(H)ₘ or N(H)ₙ
X⁸ is CH₂ or O;
X⁹ and X¹⁰ are each independently CH or N;
R²⁰, R²⁴ and R²⁵ are each independently absent, halogen, hydroxyl, alkyl, alkoxy, or haloalkyl;
R²¹ and R²² are each independently absent, —N(R²⁰)₂, halogen, hydroxyl, alkyl, haloalkyl, alkoxy;
R²³ is O, —N(R¹⁸)₂, halogen, hydroxyl, alkyl, haloalkyl, or alkoxy;
each R²⁶ is independently H, halogen, hydroxyl, alkyl, alkoxy, or haloalkyl; and
n³ is 0 or 1.

In some embodiments, the compound can be formulated in a pharmaceutical composition with a pharmaceutically acceptable carrier or excipient.

In some embodiments, the compound specifically binds to and/or complexes with an intracellular fragment or portion of an RPTP cell adhesion molecule, such as PTPμ, that is expressed by a cancer cell or another cell in the cancer cell microenvironment.

In some embodiments, the composition can be for use in detecting, monitoring, and/or imaging cancer cells and/or cancer cell metastasis, migration, dispersal, and/or invasion, and/or for treating cancer in a subject.

In other embodiments, the compositions is configured for in vivo administration to a subject or ex vivo administration to a biological sample of the subject.

In some embodiments, the compound further includes a detectable moiety linked to and/or complexed with the compound. The detectable moiety can include, for example, at least one of a contrast agent, imaging agent, radiolabel, semiconductor particle, or nanoparticle.

In some embodiments, the detectable moiety is detectable by at least one of magnetic resonance imaging positron emission tomography (PET) imaging, computer tomography (CT) imaging, gamma imaging, near infrared imaging, or fluorescent imaging.

In some embodiments, the compound can inhibit glioma cell migration in a scratch wound healing assay at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to glioma cells administered DMSO.

In other embodiments, the compound can inhibit aggregation of glioma sphere formation at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to glioma cells administered DMSO.

In still other embodiments, the compound can inhibit aggregation of PTPμ expressing SFF9 cells at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to PTPμ expressing SFF9 cells administered DMSO.

In other embodiments, the compound can inhibit PTPμ's enzymatic activity in an in vitro phosphatase assay at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% in the assay compared DMSO.

In some embodiments, the compound can inhibit tumor growth at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% in the assay compared DMSO.

### BRIEF DESCRIPTION OF THE DRAWINGS

FIGS. 1(A-D) illustrate the identification of the D2 wedge binding pocket. (A) The sequence of protein tyrosine phosphatase mu (PTPμ; UniProt P28827-1) (SEQ ID NO: 1). The amino acids that comprise the surface of the binding pocket are underlined. The linker region between the D1 and D2 domains is indicated in grey and the position of the catalytic cysteine in domain 1 is starred. (B). The domain structure of PTPμ with the D1 and D2 phosphatase domains in dark grey and pale grey, respectively, and the known regulatory wedge motif indicated in blue. This color convention is maintained throughout the figure. (C). A model for the D2 domain of PTPμ relative to the D1 region. The domains are closely associated except for a deep pocket that sits beneath a hook-shaped projection of the D2 domain. (D). Ribbon structure of the D1 and D2 domains surrounding a space filling model (red) showing the shape of the binding pocket.

FIG. 2 illustrates functional screening approach. Atomwise provided 75 compounds computationally selected for their potential to interact with the D2 binding pocket (D2BP) and two-blinded DMSO samples. The compounds were screened at 100 μM in scratch wound healing assays using two glioma cell lines (LN229 and U87). A non-blinded DMSO control was used for normalization purposes in all assays. Active compounds (and some inert controls) were tested for their ability to affect migration of an additional glioma cell line (Gli36) and for their ability to alter LN229 and Gli36 sphere formation and growth. Compounds were eliminated due to insolubility or non-specific toxicity (based on their ability to kill parental Sf9 cells, which lack PTPμ). Four compounds that were activators in some assays and inhibitors in others were not considered further, while six non-toxic glioma cell inhibitors and three glioma cell activators were tested in a highly specific assay for PTPμ-dependent adhesion (Sf9-PTPμ aggregation). These assays identified two pan-inhibitors (affecting every cell type and assay) and one activator (that stimulated U87 migration and PTPμ-dependent aggregation).

FIG. 3 illustrates the effects of all soluble D2BP compounds on LN229 scratch wound closure. Start and endpoint scratch-wound diameters were used to calculate migration distances, which were normalized to the average distance migrated by cells in the non-blinded DMSO control samples. Data are presented as average percentages ±standard errors of the means (s.e.m.). Compound bar codes are shown on the x-axis. N=2 replicates for most compounds. Some priority hits were screened with N=3-8. Endpoint images of scratch wounds treated with DMSO and two high-priority inhibitors are shown. The values indicate the relative migration distances of the selected examples.

FIG. 4 illustrates the effects of all selected compounds on Gli36 scratch wound closure. Active compounds from the initial LN229 and/or U87 screens as well as some selected controls were retested at 100 μM for effects on Gli36 migration. The average % movement of the samples relative to the non-blinded DMSO control is shown ±s.e.m. Compound bar codes are displayed on the x-axis. N=2 replicates for most compounds. Some priority hits were screened with N=4-8 replicates. Endpoint images of scratch wounds treated with DMSO and two high-priority inhibitors are shown. The values indicate the relative migration distances of the selected examples.

FIG. 5 illustrates the effects of selected compounds on LN229 sphere formation and growth. Compounds that affected LN229 and/or U87 migration as well as some selected inactive compounds were retested for effects on glioma cell (LN229) sphere formation and growth. LN229 cells cultured on non-adherent surfaces were treated with compounds (100 μM) or DMSO (1%). The footprint areas of the resulting aggregates were measured on Day 1 and Day 7. Day 1 footprint areas ≥120% that of the controls indicate inhibition of aggregation. Sphere growth was assessed by calculating the change in footprint sizes between Day 1 and Day 7 and normalizing that to the average size change of the DMSO controls. Samples showing ≤60% growth on Day 7 were deemed to be inhibited. Growth could not be calculated for samples that fell apart on Day 1 or during the assay, and this is indicated as 0% growth. Error bars are s.e.m. Most compounds were tested with n=2. Selected compounds were tested with 4-6 replicates. Examples of Day 1 and Day 7 images of samples treated with DMSO and two high-priority inhibitors are shown. Relative Day 1 footprint areas and Day 7 growth measurements are indicated for each example.

FIG. 6 illustrates the effects of selected compounds on Gli36 sphere formation and growth. Compounds that affected LN229 and/or U87 migration as well as some inactive control compounds were tested at 100 μM for effects on Gli36 cell sphere formation and growth. Sphere footprint areas were measured on Day 1 and Day 7. The Day 1 data are presented as the average % footprint size relative to the average size of the DMSO controls. The Day 7 data are presented as the average % footprint size change relative to the size change of the DMSO controls. On Day 1, footprint areas ≥120% indicate slowed aggregation, and on Day 7, values ≤indicate reduced growth. The growth of samples that fell apart on Day 1 or during the assay could not be measured, and this is indicated as 0% growth. Error bars are s.e.m. of 2-4 replicates. Representative images of samples treated with DMSO and two high-priority inhibitors are shown. Relative Day 1 footprint areas and Day 7 growth measurements are given for each example.

FIG. 7 illustrates Sf9-PTPμ aggregation assays. Sf9 cells, which lack RPTPIIb family members, were infected with baculovirus encoding full-length human PTPμ and cultured for 48 h. Cells were then treated for 20 min with compounds (at 100 μM) or DMSO and induced to aggregate by rotation. The entire surface area of each well was imaged as a 4×4 grid, aggregates >4000 μm² were counted and the values normalized to the average number present in the DMSO-treated controls. Data are presented as percentages ±s.e.m. of the indicated number of replicates. Representative images (central frames) of a DMSO control and samples treated with two priority inhibitors and one priority activator are shown. The relative number of aggregates for each example is shown.

FIG. 8 illustrates LN229 scratch wounds treated with DMSO or selected lower priority inhibitors. The relative migration distances (% DMSO control) are shown for each example.

FIG. 9 illustrates the effects of all soluble D2BP compounds on U87 scratch wound closure. Migration was quantified from start and end-point scratch wound widths and is presented as the normalized % migration ±s.e.m. relative to the DMSO controls. Compound bar codes are shown on the x-axis. All compounds were screened with an n of 2. Examples of samples treated with DMSO or two high priority inhibitors are shown.

FIG. 10 illustrates endpoint images of U87 scratch wounds treated with DMSO or selected lower priority compounds. The distance moved relative to controls for each example is indicated.

FIG. 11 illustrates example endpoint images of Gli36 scratch wounds treated with lower priority inhibitors. The distance moved relative to controls is indicated for each example.

FIG. 12 illustrates titration of selected compounds on LN229 sphere formation and growth. LN229 cells plated on non-adherent surfaces were treated with the indicated doses of compounds. The initial hit at 100 μM and the follow-up titration (at 100 μM, 50 μM, and 25 μM) are shown. Sphere footprint areas were measured on day 1 and day 7. Day 1 data is presented as the normalized sphere footprint area (% DMSO) ±. s.e.m. of two replicates. Day 7 data is presented as the normalized size change (% DMSO) in sphere footprint areas ±s.e.m. of two replicates. For samples that fell apart on day 1 or during the assay, no growth could be measured, and this is indicated as 0%. Example images of samples treated with two priority inhibitors are presented and their relative day 1 footprint areas and day 7 growth are indicated.

FIG. 13 illustrate examples of LN229 and Gli36 spheres cultured in the presence of DMSO or the indicated lower priority inhibitors (100 μM). The relative day 1 footprint area and day 7 size change for each compound are indicated.

FIG. 14 illustrates testing for compounds that affect cell viability. LN229 cells cultured on non-adherent surfaces or parental Sf9 cells (which lack PTPμ) were treated for 24 h with the indicated compounds (100 μM). Samples were then stained with Helix Blue to detect dying cells. Two compounds showed non-specific toxicity to Sf9 cells and were eliminated from the screen. The six prioritized inhibitors did not induce cell death in glioma cells or in Sf9 cells. Examples of two untreated LN229 spheres are shown because there is variability in the level of Helix Blue staining among control samples.

FIG. 15 illustrate examples of insoluble compounds. Five compounds were eliminated from the screen due to insolubility at 100 μM. This effect was more obvious in the sphere assay where black material/dust accumulated within and around spheres at the bottom of the well. 247679046 produced visible puncta in the scratch assay. In the sphere assay, these puncta appeared to nucleate the formation of multiple clumps. 247706561 precipitated in scratch assays and was not tested on spheres.

FIGS. 16(A-B) illustrate carbon skeletons and docking poses for the highest priority inhibitors. A. The carbon skeleton (CS) of 247678984 and the best docking poses from MCule 1-Click docking (https://mcule.com/apps/1-click-docking/) using Thr1071 as the binding center. The predicted Gibb's free energy of binding is −9.3 kcal/mol. B. The carbon skeleton of 246493203 and its best docking pose. The predicted Gibb's free energy of binding is −9.4 kcal/mol.

FIGS. 17(A-B) illustrate titration of selected compounds on LN229 sphere formation and growth. LN229 cells plated on non-adherent surfaces were treated with the indicated doses of compounds. The initial hit at 100 μM and the follow-up titration (at 100 μM, 50 μM, and 25 μM) are shown. Sphere footprint areas were measured on day 1 (A) and day 7 (B). Day 1 data is presented as the normalized sphere footprint area (% DMSO) ±. s.e.m. of two replicates. Day 7 data is presented as the normalized size change (% DMSO) in sphere footprint areas ±s.e.m. of two replicates. Sphere footprint areas were measured on day 1. Day 1 footprint areas ≥120% that of the controls indicate inhibition of aggregation, and on Day 7, values ≤indicate reduced growth.

FIG. 18 illustrates Sf9-PTPμ aggregation assays. Sf9 cells, which lack RPTPIIb family members, were infected with baculovirus encoding full-length human PTPμ and cultured for 48 h. Cells were then treated for 20 min with compounds (at 100 μM) or DMSO and induced to aggregate by rotation. The entire surface area of each well was imaged as a 4×4 grid, aggregates >4000 μm² were counted and the values normalized to the average number present in the DMSO-treated controls. Data are presented as percentages ±s.e.m. of the indicated number of replicates. Representative images (central frames) of a DMSO control and samples treated with two priority inhibitors and one priority activator are shown. The relative number of aggregates for each example is shown.

### DETAILED DESCRIPTION

While the following terms are believed to be well understood by one of ordinary skill in the art, the following definitions are set forth to facilitate explanation of the presently disclosed subject matter.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “or” as used herein should be understood to mean “and/or”, unless the context clearly indicates otherwise.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C_{1z} alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc.) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom, such as oxygen in oxo, carbonyl, carboxyl, and ester groups; and nitrogen in groups such as imines, oximes, hydrazones, and nitriles. For example, “substituted” includes any of the above groups in which one or more hydrogen atoms with —NRgRh, —NRgC(═O)Rh, —NRgC(═O)NRgRh, —NRgC(═O)ORh, —NRgSO2Rh, —OC(═O) NRgRh, —ORg, —SRg, —SORg, —SO₂Rg, —OSO₂Rg, —SO₂ORg, ═NSO₂Rg, and —SO₂NRgRh. “Substituted” also means any of the above groups in which one or more hydrogen atoms are replaced with —C(═O)Rg, —C(═O)ORg, —C(═O)NRgRh, —CH₂SO₂Rg, —CH₂SO₂NRgRh. In the foregoing, Rg and Rh are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

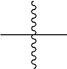

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

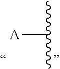

indicates that the chemical entity “A” is bonded to another chemical entity via the point of attachment bond. Furthermore, the specific point of attachment to the non-depicted chemical entity can be specified by inference. For example, the compound

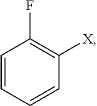

wherein X is

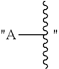

infers that the point of attachment bond is the bond by which X is depicted as being attached to the phenyl ring at the ortho position relative to fluorine.

The term “agent” is used herein to denote a chemical compound, a mixture of chemical compounds, a biological macromolecule, or an extract made from biological materials.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

The terms “patient”, “subject”, “mammalian host,” and the like are used interchangeably herein, and refer to mammals, including human and veterinary subjects.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Embodiments described herein relate to compounds that target receptor protein tyrosine phosphatase (RPTP) cell adhesion molecules (e.g., PTPμ), and particularly a binding pocket at an interface between the D1 and D2 domains of an intracellular portion or fragment of an RPTP, and that are capable of inhibiting RPTP mediated adhesion of cells and/or cancer cell growth and/or sphere formation and/or phosphatase activity as well as to their use in methods of treating cancer in a subject in need thereof and methods of detecting, monitoring, and/or imaging cancer cells and/or cancer cell metastasis, migration, dispersal, and/or invasion in a subject.

PTPmu (PTPμ) is a member of the receptor protein tyrosine phosphatase IIb family that participates in both homophilic cell-cell adhesion and signaling. PTPmu is proteolytically downregulated in glioblastoma generating extracellular and intracellular fragments that have oncogenic activity. The intracellular fragments, in particular, are known to accumulate in the cytoplasm and nucleus where they interact with inappropriate binding partners/substrates generating signals required for glioma cell migration and growth. Compounds targeting and/or interfering with these fragments can have therapeutic potential.

We used a deep learning neural network for drug design and discovery, to screen a molecular library of several million compounds and identified candidates predicted to interact with a binding pocket bordered by the wedge domain, a known regulatory motif located within the juxtamembrane portion of the protein. These candidates were then screened in multiple cell-based assays for effects on glioma cell motility (scratch assays) and growth in 3D culture (sphere assays), and PTPmu-dependent adhesion (Sf9 aggregation). Compounds that that affected the motility of multiple glioma cell lines (LN229, U87MG, and Gli36delta5), the growth of LN229 and Gli36 spheres, PTPmu-dependent Sf9 aggregation and/or suppressed PTPmu enzymatic activity in an in vitro phosphatase assay, and/or inhibited the growth of human glioma tumors in mice can be used as a therapeutic agent to reduce cancer cell, e.g., glioma cell or glioblastoma, growth, invasion, and/or metastasis. Additionally, such compounds can be used as a targeted molecular imaging agent in brain tumor diagnosis and/or as a targeted optical imaging agent in fluorescent guided surgical resection of brain tumors.

For example, when the compound includes a detectable moiety that is directly or indirectly linked to the compound, the compound can demarcate tumor cells in tissue sections and tumor “edge” samples, suggesting that the compound can be used as a diagnostic tool for molecular imaging of metastatic, dispersive, migrating, or invading cancers or the tumor margin. Systemic introduction of compounds as described herein can result in specific labeling of the tumors.

The compounds described herein can be administered systemically to a subject and readily target cancer cells associated with proteolytically cleaved intracellular fragment of the RPTP type IIb cell adhesion molecules, such as metastatic, migrating, dispersed, and/or invasive cancer cells. In some embodiments, the compounds after systemic administration can cross the blood brain barrier to define cancer cell location, distribution, metastases, dispersions, migrations, and/or invasion as well as tumor cell margins in the subject. In other embodiments, the compounds after systemic administration can inhibit and/or reduce cancer cell growth, survival, proliferation, and migration.

The compounds described herein can therefore be used in a method of inhibiting cancer cell metastasis, migration, dispersal, and/or invasion as well as in a method of treating cancer in a subject in need thereof. The methods can include administering to a subject a compound that binds to and/or complexes with a binding pocket adjacent a wedge domain of an intracellular portion or fragment of the RPTP cell adhesion molecule in the cancer cell or tumor cell microenvironment. The compound bound to and/or complexed with the binding pocket adjacent the wedge domain of the intracellular portion or fragment of RPTP cell adhesion molecule expressed by the cancer cells can inhibit and/or reduce cancer cell growth, survival, proliferation, and/or migration as well as can be detected to determine the location and/or distribution of the cancer cells in the subject.

In some embodiments, the compound can have the structure of formula (I):

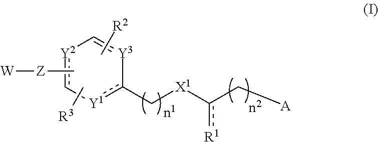

or a pharmaceutically acceptable salt, tautomer, or solvate thereof,
wherein:
a dashed line (e.g., ---- or --) is an optional bond;
A is cycloalkyl, aryl, heteroaryl, or heterocyclyl, each of which is optionally substituted with one or more R⁴;
X¹ is CH₂ or O;
Y¹, Y², and Y³ are each independently C(H)ₘ or N(H)ₙ;
W is absent or an aryl or heteroaryl, each optionally substituted with one or more R^{3a};
Z is absent or an alkylene, -alkylene-cycloalkylene-, -alkylene-heterocyclylene-, or —N(R⁶)—, each optionally substituted with one or more R⁵;
R¹ and R⁵ are each independently O, —N(R⁶)₂, halogen, hydroxyl, alkyl, haloalkyl, or alkoxy;
R², R³, and each R^{3a} are each independently absent, —N(R⁶)₂, halogen, hydroxyl, alkyl, haloalkyl, alkoxy;
R⁴ is ═O, halogen, hydroxyl, alkyl, alkoxy, haloalkyl, aryl, or haloaryl;
R⁶ is H, halogen, hydroxyl, alkyl, alkoxy, or haloalkyl;
m is 0, 1, or 2;
n and n¹ are each independently 0 or 1; and
n² is 0, 1, 2, or 3.

In some embodiments, A is 5- to 6-membered cycloalkyl or 5- to 10-membered aryl or heteroaryl, each of which is optionally substituted with one or more R⁴.

In some embodiments, A is a 5- to 6-membered heterocyclyl or 5- to 10-membered heteroaryl, each of which is optionally substituted with one or more R⁴.

In other embodiments, A is a 5- to 6-membered heteroaryl optionally substituted with one or more R⁴.

In still other embodiments, A is an 8- to 10-membered bicyclic heteroaryl optionally substituted with one or more R⁴.

In some embodiments, A is a dihydro-indenyl, indenyl, indolinyl, indolyl, isoindolyl, indolizinyl, indazolyl, benzimidazolyl, azaindolyl, pyrazolo-pyrimidinyl, purinyl, benzofuranyl, isobenzofuranyl, or benzothiophenyl, each of which is optionally substituted with one or more R⁴.

In other embodiments A is selected from:

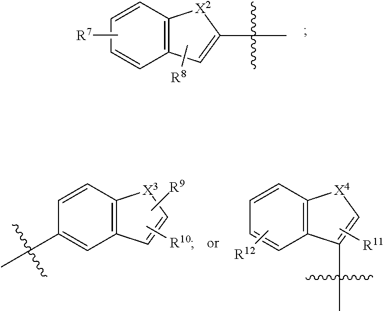

wherein X², X³, and X⁴ are each independently N(R¹³) or CH₂,
R⁷, R⁸, R⁹, and R¹⁰ are each independently absent, halogen, hydroxyl, alkyl, alkoxy, or haloalkyl;
R¹¹ and R¹² are each independently absent, halogen, hydroxyl, alkyl, alkoxy, haloalkyl, aryl or haloaryl; and
R¹³ is H, halogen, hydroxyl, alkyl, alkoxy, or haloalkyl.

In some embodiments, R⁷, R⁸, R⁹, and R¹⁰ are a C₁-C₆ alkyl or C₁-C₆ haloalkyl.

In some embodiments, R¹¹ is 5- to 10-membered aryl or haloaryl, and R¹² is absent or a halogen.

In some embodiments, X² is N(R¹³) and R⁷ and R⁸ are each independently absent, halogen, hydroxyl, C₁-C₆ alkyl, C₁-C₆ alkoxy, or C₁-C₆ haloalkyl.

In some embodiments, X³ is N(R¹³) and R⁹ and R¹⁰ are each independently absent, halogen, hydroxyl, C₁-C₆ alkyl, C₁-C₆ alkoxy, or C₁-C₆ haloalkyl.

In some embodiments, X⁴ is N(R¹³) and R¹¹ is absent, 5- to 10-membered cyclic or bicyclic aryl or haloaryl and R¹² is absent, a halogen, or C₁-C₆ alkyl.

In some embodiments, A is selected from

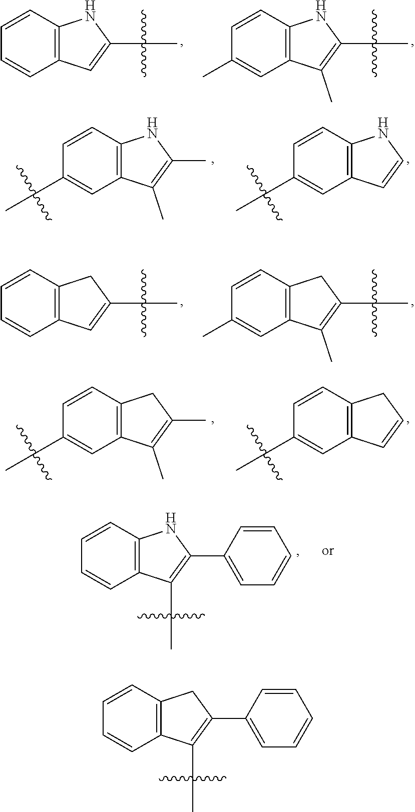

each of which is optionally substituted with one or more halogen.

In some embodiments, R¹ and R⁵ are each independently O, —N(R⁶)₂, halogen, hydroxyl, C₁-C₆ alkyl, C₁-C₆ haloalkyl, or C₁-C₄ alkoxy.

In some embodiments, R², R³, and R^{3a} are each independently absent, —N(R⁶)₂, halogen, hydroxyl, C₁-C₆ alkyl, C₁-C₆ haloalkyl, or C₁-C₄ alkoxy;

In some embodiments, R⁴ is ═O, halogen, hydroxyl, C₁-C₆ alkyl, C₁-C₄ alkoxy, C₁-C₆ haloalkyl, 5- to 10-membered aryl, or 5- to 10-membered haloaryl;

In some embodiments, R⁶ is H, halogen, hydroxyl, C₁-C₆alkyl, C₁-C₄ alkoxy, or C₁-C₆ haloalkyl.

In some embodiments, R¹ is O.

In some embodiments, X¹ is O.

In some embodiments, n¹ is 1; n² is 0; Y¹, Y², and Y³ are each independently N; and R² is —N(R⁶)₂, preferably, —NH₂, and R³ is absent.

In some embodiments, n¹ is 0; n² is 0; Y¹, Y², and Y³ are each independently CH; and R² and R³ are absent.

In some embodiments, Z is a C₁-C₈ alkylene, —(C₁-C₄ alkylene)-(4- to 6-membered cycloalkylene)-, —(C₁-C₄ alkylene)-(4- to 6-membered heterocyclylene)-, or —N(R⁶)-aryl, each optionally substituted with one or more R⁵.

In some embodiments, Z is:

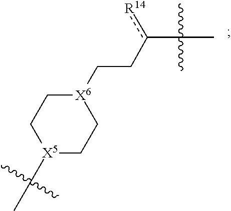

### or —N(H)—;

wherein
X⁵ and X⁶ are each independently CH or N; R¹⁴ is O, —N(H)₂, halogen, hydroxyl, alkyl, haloalkyl, or alkoxy; and W is a 6- to 8-membered aryl or 5- to 6-membered heteroaryl, each optionally substituted with one or more R^{3a}.

In other embodiments, Z is:

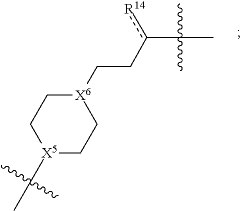

X⁵ and X⁶ are each independently CH or N, preferably N; and R¹⁴ is O; W is a 6- to 8-membered aryl or 5- to 6-membered heteroaryl, and R³ is absent, halogen, or alkoxy.

In some embodiments, Z is —N(H)—; W is a 6- to 8-membered aryl or 5- to 6-membered heteroaryl; and R^{3a} is absent.

In some embodiments, n¹ is 1; n² is 1, 2, or 3; Y¹, Y², and Y³ are each independently N; and W and Z are absent. R² and R³ can each independently be —N(R⁶)₂, preferably R⁶ is H.

In some embodiments, A is

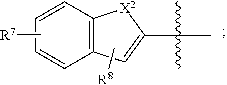

n¹ is 0;
n² is 0;
X¹ is 0;
X² is N(R¹³);
Y¹, Y², and Y³ are each independently C(H);
Z is

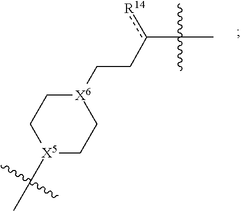

W is a 6- to 8-membered aryl optionally substituted with one or more R^{3a};
X⁵ and X⁶ are each independently N;
R¹ is O;
R² and R³ are absent;
each R^{3a} is absent, halogen, alkyl, or alkoxy;
R⁷ and R⁸ are each independently absent, halogen, hydroxyl, alkyl, alkoxy, or haloalkyl, preferably an alkyl, and more preferably a C₁-C₆ alkyl;
R¹³ is H, halogen, hydroxyl, alkyl, alkoxy, or haloalkyl; and
R¹⁴ is O, —N(H)₂, halogen, hydroxyl, alkyl, haloalkyl, or alkoxy, preferably O.

In other embodiments,
A is

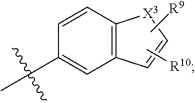

n¹ is 1;
n² is 0;
X¹ is O;
X³ is N(R¹³);
Y¹, Y², and Y³ are each independently N;
Z is —N(H)—;
W is a 6- to 8-membered aryl optionally substituted with one or more R^{3a};
R¹ is O;
R² and R³ are each independently absent, —N(R⁶)₂, halogen, hydroxyl, alkyl, haloalkyl, alkoxy;
R^{3a} is absent, halogen, alkyl, or alkoxy;
R⁶ is H, halogen, hydroxyl, alkyl, alkoxy, or haloalkyl;
R⁹ and R¹⁰ are each independently absent, halogen, hydroxyl, alkyl, alkoxy, or haloalkyl, preferably an alkyl, and more preferably a C₁-C₆ alkyl; and
R¹³ is H, halogen, hydroxyl, alkyl, alkoxy, or haloalkyl.

In still other embodiments,
A is

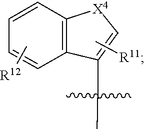

n¹ is 1;
n² is 1, 2, or 3;
X¹ is O;
X⁴ is N(R¹³);
Y¹, Y², and Y³ are each independently N;
Z and W are absent;
R¹ is O;
R² and R³ are each independently absent, —N(R⁶)₂, halogen, hydroxyl, alkyl, haloalkyl, alkoxy;
R⁶ is H, halogen, hydroxyl, alkyl, alkoxy, or haloalkyl;
R¹¹ and R¹² are each independently absent, halogen, hydroxyl, alkyl, alkoxy, haloalkyl, aryl or haloaryl, and preferably R¹² is a halogen and R¹¹ is an aryl or haloaryl; and
R¹³ is H, halogen, hydroxyl, alkyl, alkoxy, or haloalkyl.

Other embodiments described herein relate to pharmaceutical composition that includes a compound of formula (IA):

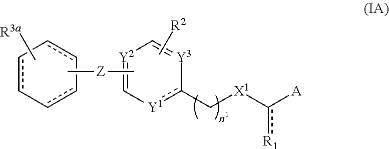

or a pharmaceutically acceptable salt, tautomer, or solvate thereof,
wherein:
a dashed line (e.g., ---- or --) is an optional bond;
A is cycloalkyl, aryl, heteroaryl, or heterocyclyl, each of which is optionally substituted with one or more R⁴;
X¹ is CH₂ or O;
Y¹, Y², and Y³ are each independently C(H)ₘ or N(H)ₙ;
Z is an alkyl, -alkyl-cycloalkyl-, -alkyl-heterocyclyl-, or —N(R⁶)—, each optionally substituted with one or more R⁵,
R¹ and R⁵ are each independently O, —N(R⁶)₂, halogen, hydroxyl, alkyl, haloalkyl, or alkoxy;
R² and each R^{3a} are each independently absent, —N(R⁶)₂, halogen, hydroxyl, alkyl, haloalkyl, alkoxy;
R⁴ is ═O, halogen, hydroxyl, alkyl, alkoxy, or haloalkyl;
R⁶ is H, halogen, hydroxyl, alkyl, alkoxy, or haloalkyl;
m is 0, 1, or 2; and
n and n¹ are each independently 0 or 1.

In some embodiments, A is a dihydro-indenyl, indenyl, indolinyl, indolyl, isoindolyl, indolizinyl, indazolyl, benzimidazolyl, azaindolyl, pyrazolo-pyrimidinyl, purinyl, benzofuranyl, isobenzofuranyl, benzothiophenyl, each of which is optionally substituted with one or more R⁴.

In some embodiments, A is

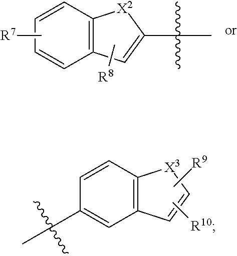

X² and X³ are each independently N(R¹³) or CH₂; R⁷, R⁸, R⁹, and R¹⁰ are each independently absent, halogen, hydroxyl, alkyl, alkoxy, or haloalkyl; and R¹³ is H, halogen, hydroxyl, alkyl, alkoxy, or haloalkyl.

In some embodiments, X² is N(R¹³).

In some embodiments, X³ is N(R¹³).

In other embodiments, A is selected from

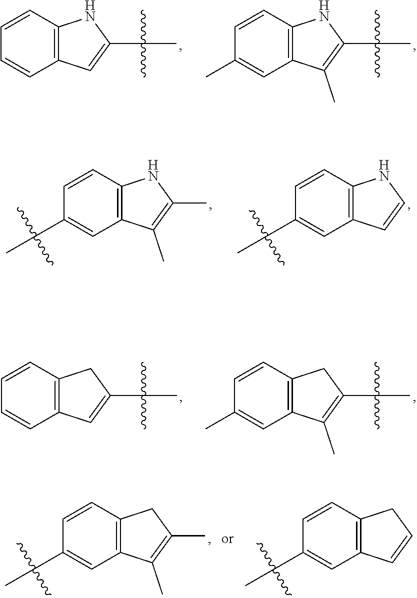

each of which is optionally substituted with one or more halogen.

In some embodiments, R¹ is O.

In some embodiments, X¹ is O.

In some embodiments, n¹ is 1 and Y¹, Y², and Y³ are each independently N. R² is —N(R⁶)₂, preferably, —NH₂.

In some embodiments, n¹ is 0 and Y¹, Y², and Y³ are each independently CH. R² is absent.

In some embodiments, Z is:

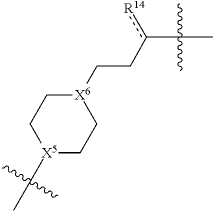

or —N(H)—; X⁵ and X⁶ are each independently CH or N; and R¹⁴ is O, —N(H)₂, halogen, hydroxyl, alkyl, haloalkyl, or alkoxy.

In other embodiments, Z is:

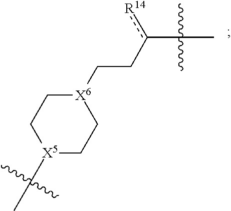

X⁵ and X⁶ are each independently CH or N, preferably N; R¹⁴ is O; and R³ is absent, halogen, or alkoxy.

In still other embodiments, Z is —N(H)—, and R^{3a} is absent.

Still other embodiments relate to a pharmaceutical composition that includes a compound of formula (II):

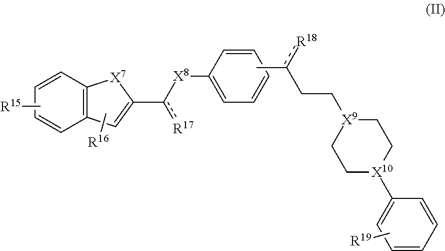

or a pharmaceutically acceptable salt, tautomer, or solvate thereof,
wherein,
a dashed line (e.g., ---- or --) is an optional bond;
X⁷ is N(R²⁰) or CH₂;
X⁸ is CH₂ or O;
X⁹ and X¹⁰ are each independently CH or N;
R¹⁵ and R¹⁶ are each independently absent, halogen, hydroxyl, alkyl, alkoxy, or haloalkyl;
R¹⁷ and R¹⁸ are each independently O, —N(R²⁰)₂, halogen, hydroxyl, alkyl, haloalkyl, or alkoxy;
R¹⁹ is absent, —N(R²⁰)₂, halogen, hydroxyl, alkyl, haloalkyl, alkoxy; and
each R²⁰ is independently H, halogen, hydroxyl, alkyl, alkoxy, or haloalkyl.

In some embodiments, X⁷ is N(R²⁰); X⁸ is O; X⁹ and X¹⁰ are N; R¹⁵ and R¹⁶ are each independently absent, halogen, hydroxyl, C₁-C₆ alkyl, C₁-C₆ alkoxy, or C₁-C₆ haloalkyl; R¹⁷ and R¹⁸ are each independently O; R¹⁹ is absent, halogen, or alkoxy; and R²⁰ is H, halogen, C1-C₆ alkyl, or C₁-C₆ haloalkyl.

Still other embodiments relate to a pharmaceutical composition that includes a compound of formula (III):

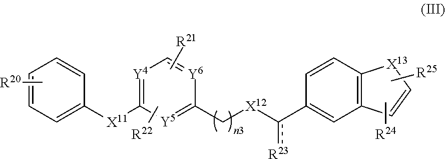

or a pharmaceutically acceptable salt, tautomer, or solvate thereof;
wherein:
a dashed line (e.g., ---- or --) is an optional bond;
X¹¹ and X¹³ are each independently N(R²⁶) or CH₂;
X¹² is CH₂ or O;
Y⁴, Y⁵, and Y⁶ are each independently C(H)ₘ or N(H)ₙ
X⁸ is CH₂ or O;
X⁹ and X¹⁰ are each independently CH or N;
R²⁰, R²⁴ and R²⁵ are each independently absent, halogen, hydroxyl, alkyl, alkoxy, or haloalkyl;
R²¹ and R²² are each independently absent, —N(R²⁰)₂, halogen, hydroxyl, alkyl, haloalkyl, alkoxy;
R²³ is O, —N(R¹⁸)₂, halogen, hydroxyl, alkyl, haloalkyl, or alkoxy;
each R²⁶ is independently H, halogen, hydroxyl, alkyl, alkoxy, or haloalkyl; and
n³ is 0 or 1.

In other embodiments, the compound can be selected from:

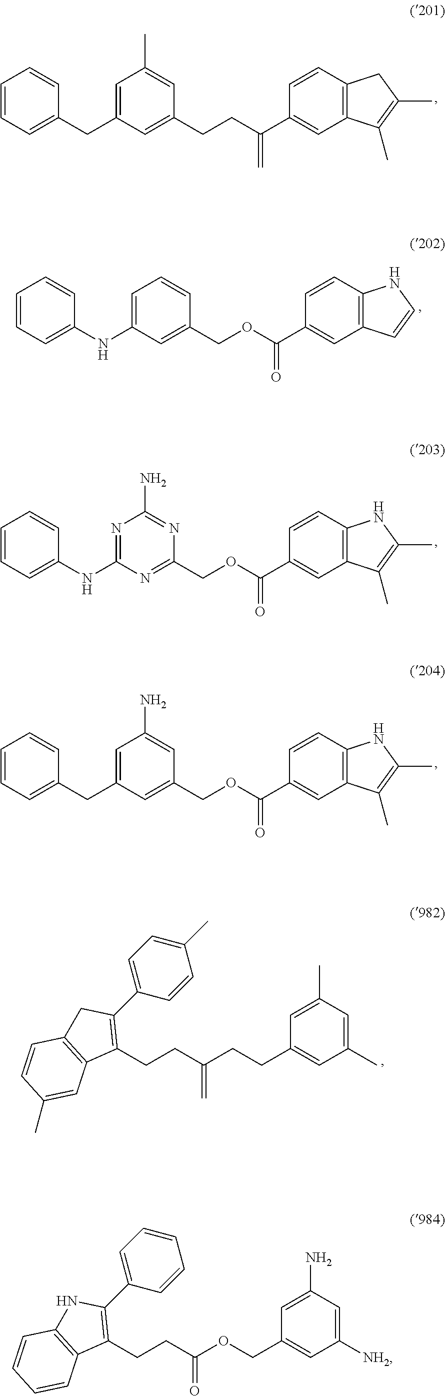

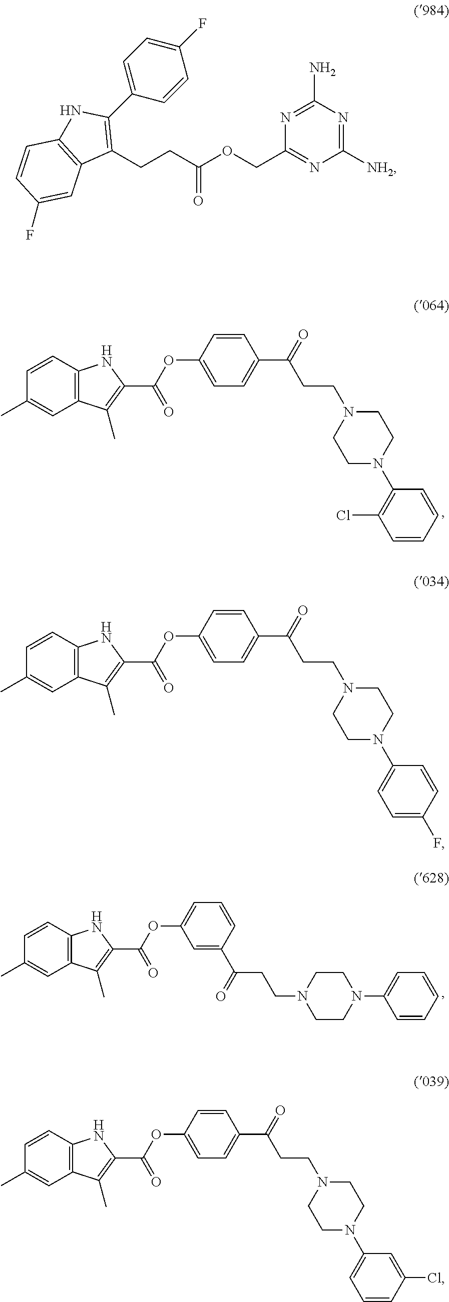 [SMILES: Nc1nc(N)nc(COC(=O)CCc2c(-c3ccc(F)cc3)[nH]c3ccc(F)cc23)n1 ; Cc1ccc2[nH]c(C(=O)Oc3ccc(C(=O)CCN4CCN(c5ccccc5Cl)CC4)cc3)c(C)c2c1 ; Cc1ccc2[nH]c(C(=O)Oc3ccc(C(=O)CCN4CCN(c5ccc(F)cc5)CC4)cc3)c(C)c2c1 ; Cc1ccc2[nH]c(C(=O)Oc3cccc(C(=O)CCN4CCN(c5ccccc5)CC4)c3)c(C)c2c1 ; Cc1ccc2[nH]c(C(=O)Oc3ccc(C(=O)CCN4CCN(c5cccc(Cl)c5)CC4)cc3)c(C)c2c1]

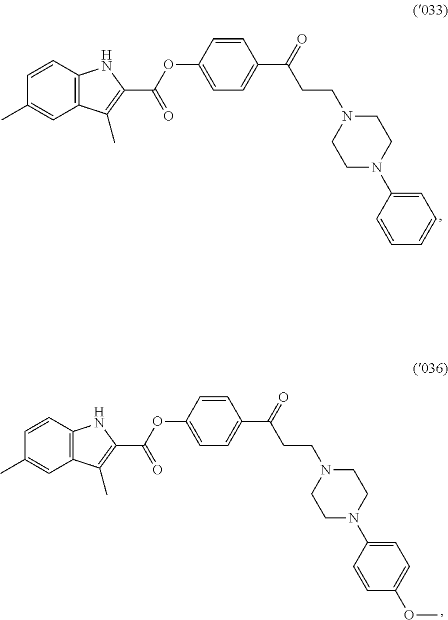

or a pharmaceutically acceptable salt, tautomer, or solvate thereof.

In other embodiments, the compound can be selected from:

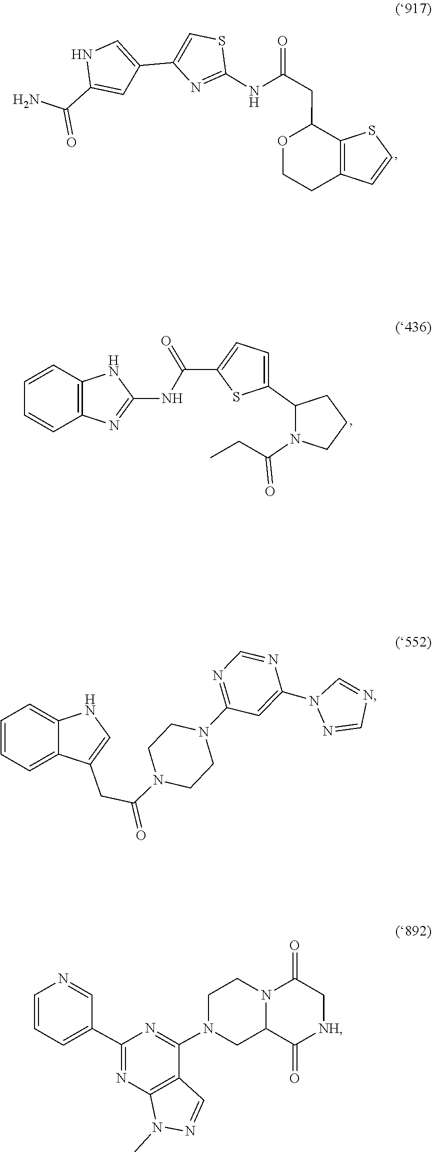 [SMILES: NC(=O)c1cc(-c2csc(NC(=O)CC3OCCc4ccsc43)n2)c[nH]1 ; CCC(=O)N1CCCC1c1ccc(C(=O)Nc2nc3ccccc3[nH]2)s1 ; O=C(Cc1c[nH]c2ccccc12)N1CCN(c2cc(-n3cncn3)ncn2)CC1 ; Cn1ncc2c(N3CCN4C(=O)CNC(=O)C4C3)nc(-c3cccnc3)nc21]

or a pharmaceutically acceptable salt, tautomer, or solvate thereof.

In some embodiments, the efficacy of the compound can be assessed by generating dose response curves from data obtained using various concentrations of a test compound. Moreover, a control assay can also be performed to provide a baseline for comparison. Such candidates can be further tested for efficacy in inhibiting chemotaxis of cancer cells in vitro, spreading, invasion, or migration of cancer cells in vitro, for efficacy in tumor dispersal, or spreading in vitro or in vivo. For example, the efficacy of the compound can be tested in vivo in animal cancer models.

Cell-based assays may be performed as either a primary screen, or as a secondary screen to confirm the activity of agents identified in a cell free screen, such as an in silico screen. Such cell based assays can employ a cell-type expressing the RPTP. Exemplary cell types include cancer cell lines, primary tumor xenografts, and glioma cells. Cells in culture are contacted with one or more compounds, and the ability of the one or more compounds to inhibit cell migration/invasion is measured. Compounds that inhibit cell migration/invasion are candidate compounds for use in the subject methods of inhibiting tumor progression. For example, the identified compounds can be tested in cancer models known in the art.

In some embodiments, putative compounds identified by in silico screens can be further screened or assessed for efficacy using scratch wound healing assays with glioma cell lines. Scratch wound healing assays measure the ability of cells to migrate into a wound and close it creating a monolayer. The scratch wound healing assays can be performed using LN229, U87, and Gli36 glioma cell lines. These cell lines express different levels of full-length PTPμ and its fragments and have different invasive behaviors in orthotopic tumor models. LN229 cells express mainly PTPμ fragments and are invasive; U87 cells express full-length and some PTPμ fragments and exhibit little invasive behavior in vivo; whereas Gli36 cells have very little full-length PTPμ but express fragments and the sensitivity profile of these cells are expected to be similar to that of the LN229 cells. Soluble wedge-targeting compounds, which are identified with an in silico screen, effects on scratch wound closure can be quantified from scratch wound widths at the start and end of the assay and normalized to the average movement of cells in the unblinded DMSO control samples.

In some embodiments, about 100 μM, preferably about 50 μM, or more preferably about 25 μM of a compound described herein can inhibit glioma cell migration in such scratch wound assays at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to glioma cells administered DMSO.

In other embodiments, efficacy of the compounds can be measured using a glioma sphere assay. The glioma cell sphere formation and growth assay tests cell-cell adhesion and the ability to grow in 3-dimensions, creating a structure that more closely mimics a tumor and its microenvironment. Compounds that are active in this assay are more likely to be effective in vivo. This assay can be selected to run in parallel with scratch wound healing assays. Glioma cells (LN229s) cultured on non-adhesive coating cluster together and grow as 3D structures that can model some of the complexity of the tumor microenvironment.

In some embodiments, about 100 μM, preferably about 50 μM, or more preferably about 25 μM of a compound described herein can inhibit aggregation of glioma sphere formation at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to glioma cells administered DMSO.

In other embodiments, efficacy of the compounds can be measured using a PTPμ-dependent Sf9 aggregation assay. The Sf9 assay directly tests adhesive action of PTPμ since Sf9 cells lack endogenous PTPμ and do not normally self-aggregate. However, baculoviral-mediated overexpression of PTPμ drives homophilic adhesion of Sf9 cells on non-adhesive coated wells. PTPμ expressing Sf9 cells readily aggregate in control samples, but wells treated with therapeutically effective compounds can contain mostly single cells or small clusters.

In some embodiments, aggregation of PTPμ expressing Sf9 cells administered about 100 μM, preferably about 50 μM, or more preferably about 25 μM, of a compound described herein can be inhibited at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% compared to PTPμ expressing SFF9 cells administered DMSO.

In other embodiments, efficacy of compounds can be measured by the compounds' ability to alter PTPμ's enzymatic activity with an in vitro phosphatase assay. In the assay, a GST-tagged protein corresponding to the entire intracellular domain of human PTPμ can be preincubated with DMSO or selected compounds and then the reaction started by addition of a peptide substrate and incubation an elevated temperature (e.g., 30° C.). At the endpoint of the assay, released phosphate can be measured using a colorimetric reaction and normalized to the amount released by the vehicle-treated control.

In some embodiments, about 100 μM, preferably about 50 μM, or more preferably about 25 μM, of a compound described herein can inhibit enzymatic activity or released phosphate at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% in the assay compared DMSO.

In other embodiments, efficacy of compounds can be measured by the compounds' ability to affect tumor growth in vivo. In such an assay, human LN229 glioma cells are subcutaneously injected into the flanks of athymic nude mice. Once tumors are established, DMSO or a test compound can be injected into the center of each tumor, and tumor volumes can calculated from caliper measurements.

In some embodiments, about 100 μM, preferably about 50 μM, or more preferably about 25 μM, of a compound described herein can inhibit tumor growth at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% in the assay compared DMSO.

In other embodiments, the compounds can include or be directly or indirectly coupled to a detectable moiety. The detectable moiety can include any contrast agent or detectable label that facilitate the detection step of a diagnostic or therapeutic method by allowing visualization of the complex formed by binding of the compound to the intracellular portion or fragment of the RPTP cell adhesion molecule. The detectable moiety can be selected such that it generates a signal, which can be measured and whose intensity is related (preferably proportional) to the amount of the compound bound to the tissue being analyzed.

Any of a wide variety of detectable moieties can be linked with the compounds described herein. Examples of detectable moieties include, but are not limited to: various ligands, radionuclides, fluorescent agents and dyes, infrared and near infrared agents, chemiluminescent agents, microparticles or nanoparticles (e.g., quantum dots, nanocrystals, semiconductor particles, nanoparticles, nanobubbles, or nanochains and the like), colorimetric labels, magnetic labels, and chelating agents.

In some embodiments, compounds including the detectable moiety described herein may be used in conjunction with non-invasive imaging (e.g., neuroimaging) techniques for in vivo imaging of the compound, such as magnetic resonance spectroscopy (MRS) or imaging (MRI), or gamma imaging, such as positron emission tomography (PET) or single-photon emission computed tomography (SPECT). The term “in vivo imaging” refers to any method, which permits the detection of a labeled compound, as described above. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of the compound along with a large excess of unlabeled, but otherwise chemically identical compound.

For purposes of in vivo imaging, the type of detection instrument available is a major factor in selecting a given detectable moiety. For instance, the type of instrument used will guide the selection of the stable isotope. The half-life should be long enough so that it is still detectable at the time of maximum uptake by the target, but short enough so that the host does not sustain deleterious effects.

In one example, the detectable moiety can include a radiolabel, that is directly or indirectly linked (e.g., attached or complexed) with a compound described herein using general organic chemistry techniques. The radiolabel can be, for example, ⁶⁸Ga, ¹²³I, ¹³¹I, ¹²⁵I, ¹⁸F, ¹¹C, ⁷⁵Br, ⁷⁶Br, ¹²⁴I, ¹³N, ⁶⁴Cu, ³²P, ³⁵S. Such radiolabels can be detected by PET techniques, such as described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, NY 1986) the contents of which are hereby incorporated by reference. The detectable moiety can also include ¹²³I for SPECT. The ¹²³I can be coupled to the compound by any of several techniques known to the art. In addition, the detectable moiety can include any radioactive iodine isotope, such as, but not limited to ¹³¹I, ¹²⁵I, or ¹²³I. The radioactive iodine isotopes can be coupled to the compound, for example, by conversion of a non-radioactive halogenated precursor to a stable tri-alkyl tin derivative which then can be converted to the iodo compound by several methods well known to the art.

The detectable moiety can further include known metal radiolabels, such as Technetium-99m (99mTc), ¹⁵³Gd, ¹¹¹In, ⁶⁷Ga, ²⁰¹Tl, ⁸²Rb, ⁶⁴Cu, ⁹⁰Y, ¹⁸⁸Rh, T(tritium), ¹⁵³Sm, ⁸⁹Sr, and ²¹¹At. Modification of the compound to introduce ligands that bind such metal ions can be effected without undue experimentation by one of ordinary skill in the radiolabeling art. The metal radiolabeled compounds can then be used to detect cancers, such as GBM in the subject. Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2):135-40, (1998); and Horn et al., “Technetium-99m-labeled receptor-specific small-molecule radiopharmaceuticals: recent developments and encouraging results” Nuclear Medicine & Biology 24(6):485-98, (1997).

In some embodiments, the detectable moiety can include a chelating agent (with or without a chelated radiolabel metal group). Examples chelating agents can include those disclosed in U.S. Pat. No. 7,351,401, which is herein incorporated by reference in its entirety. In some embodiments, the chelating agent is 1,4,7,10-tetraazacyclododecane-1,4,7,10-tetraacetic acid (DOTA).

Fluorescent labeling agents or infrared agents include those known to the art, many of which are commonly commercially available, for example, fluorophores, such as ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED, BODIPY TR-X, ALEXA 594, BODIPY 630/650-X, PULSAR 650, BODIPY 630/665-X, ALEXA 647, IR700, IR800, TEXAS RED, and QUASAR 670.

In some embodiments, the detectable moiety includes a fluorescent dye. Examples of fluorescent dyes include fluorescein isothiocyanate, cyanines, such as Cy5, Cy5.5 and analogs thereof (e.g., sulfo-Cyanine 5 NHS ester and Cy5.5 maleimide). See also Handbook of Fluorescent Probes and Research Chemicals, 6th Ed., Molecular Probes, Inc., Eugene Oreg, which is incorporated herein by reference.

The detectable moiety can further include a near infrared imaging group. Near infrared imaging groups are disclosed in, for example, Tetrahedron Letters 49(2008) 3395-3399; Angew. Chem. Int. Ed. 2007, 46, 8998-9001; Anal. Chem. 2000, 72, 5907; Nature Biotechnology vol 23, 577-583; Eur Radiol (2003) 13: 195-208; and Cancer 67: 1991 2529-2537, which are herein incorporated by reference in their entirety. Applications may include the use of a NIRF (near infra-red) imaging scanner. In one example, the NIRF scanner may be handheld. In another example, the NIRF scanner may be miniaturized and embedded in an apparatus (e.g., micro-machines, scalpel, neurosurgical cell removal device).

Quantum dots, e.g., semiconductor particles, can be employed as detectable moieties as described in Gao, et al “In vivo cancer targeting and imaging with semiconductor quantum dots”, Nature Biotechnology, 22, (8), 2004, 969-976, the entire teachings of which are incorporated herein by reference. The disclosed compounds can be coupled to the quantum dots, administered to a subject or a sample, and the subject/sample examined by fluorescence spectroscopy or imaging to detect the labeled compound.

In certain embodiments, a detectable moiety includes an MRI contrast agent. MRI relies upon changes in magnetic dipoles to perform detailed anatomic imaging and functional studies. MRI can employ dynamic quantitative T1 mapping as an imaging method to measure the longitudinal relaxation time, the T1 relaxation time, of protons in a magnetic field after excitation by a radiofrequency pulse. T1 relaxation times can in turn be used to calculate the concentration of a molecular probe in a region of interest, thereby allowing the retention or clearance of an agent to be quantified. In this context, retention is a measure of molecular contrast agent binding.

Numerous magnetic resonance imaging (MRI) contrast agents are known to the art, for example, positive contrast agents and negative contrast agents. The disclosed compounds can be coupled to the MRI agents, administered to a subject or a sample, and the subject/sample examined by MRI or imaging to detect the labeled compound. Positive contrast agents (typically appearing predominantly bright on MRI) can include typically small molecular weight organic compounds that chelate or contain an active element having unpaired outer shell electron spins, e.g., gadolinium, manganese, iron oxide, or the like. Typical contrast agents include macrocycle-structured gadolinium(III)chelates, such as gadoterate meglumine (gadoteric acid), gadopentetate dimeglumine, gadoteridol, mangafodipir trisodium, gadodiamide, and others known to the art. In certain embodiments, the detectable moiety includes gadoterate meglumine. Negative contrast agents (typically appearing predominantly dark on MRI) can include small particulate aggregates comprised of superparamagnetic materials, for example, particles of superparamagnetic iron oxide (SPIO). Negative contrast agents can also include compounds that lack the hydrogen atoms associated with the signal in MRI imaging, for example, perfluorocarbons (perfluorochemicals).

In some embodiments, the compound can be coupled or linked to a chelating agent, such as macrocyclic chelator DOTA, and a single metal radiolabel.

The compounds described herein can be used in a pharmaceutical composition to detect and/or treat a variety of cancers that express RPTP including (but not limited to) the following: carcinoma, including that of the bladder, breast, prostate, rectal, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma.

In certain embodiments, cancer cells that express an RPTP can include glioma cells. The term glioma, as used herein, refers to a type of cancer arising from glial cells in the brain or spine. Gliomas can be classified by cell type, by tumor grade, and by location. For example, ependymomas resemble ependymal cells, astrocytomas (e.g., glioblastoma multiforme) resemble astrocytes, oligodendrogliomas resemble oligodendrocytes. Also mixed gliomas, such as oligoastrocytomas may contain cells from different types of glia. Gliomas can also be classified according to whether they are above or below a membrane in the brain called the tentorium. The tentorium separates the cerebrum, above, from the cerebellum, below. A supratentorial glioma is located above the tentorium, in the cerebrum, and occurs mostly in adults whereas an infratentorial glioma is located below the tentorium, in the cerebellum, and occurs mostly in children.

In still other embodiments, the cancer cells that are detected and/or treated can include invasive, dispersive, motile or metastatic cancer cells, such as invasive, dispersive, motile or metastatic glioma cells, lung cancer cells, breast cancer cells, prostate cancer cells, and melanoma cells. It will be appreciated that other cancer cells and/or endothelial cells, which support cancer cell survival, that express an RPTP cell adhesion molecule and that can be proteolytically cleaved to produce a detectable extracellular fragment can be identified or determined by, for example, using immunoassays that detect the RPTP cell adhesion molecule expressed by the cancer cells or endothelial cells.

A pharmaceutical composition that includes a compound described herein can be administered to the subject by, for example, systemic, topical, and/or parenteral methods of administration. These methods include, e.g., injection, infusion, deposition, implantation, or topical administration, or any other method of administration where access to the tissue by the molecular probe is desired. In one example, administration of the compound probe can be by intravenous injection of the compound in the subject. Single or multiple administrations of the compound can be given. “Administered”, as used herein, means provision or delivery of compound in an amount(s) and for a period of time(s) effective to label or treat cancer cells in the subject.

In some embodiments, the compounds described herein can be administered to a cancer cell, e.g., glioblastoma multiforme cell, prostate cancer, lung cancer, melanoma, or tumor-derived endothelial cell of a subject by contacting the cell of the subject with a pharmaceutical composition described above. In one aspect, a pharmaceutical composition can be administered directly to the cell by direct injection. Alternatively, the pharmaceutical composition can be administered to the subject systematically by parenteral administration, e.g., intravenous administration or oral.

In a further example, the compound can be used in combination and adjunctive therapies for inhibiting cancer cell proliferation, growth, and motility. The phrase “combination therapy” embraces the administration of the compounds described herein and an additional therapeutic agent as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). The phrase “adjunctive therapy” encompasses treatment of a subject with agents that reduce or avoid side effects associated with the combination therapy of this application.

A combination therapy is intended to embrace administration of these therapeutic agents in a sequential manner, that is, wherein different therapeutic agents are administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of therapeutic agents can be effected by an appropriate routes including, but not limited to, oral routes, intravenous routes, intramuscular routes, and direct absorption through mucous membrane tissues. The therapeutic agents can be administered by the same route or by different routes. The sequence in which the therapeutic agents are administered is not narrowly critical.

Combination therapy also can embrace the administration of the therapeutic agents as described above in further combination with other biologically active ingredients (such as, but not limited to, a second and different therapeutic agent) and non-drug therapies (such as, but not limited to, surgery or radiation treatment). Where the combination therapy further comprises radiation treatment, the radiation treatment may be conducted at a suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments the compounds described herein can be administered in combination at least one anti-proliferative agent selected from a chemotherapeutic agent, an antimetabolite, an antitumorigenic agent, an antimitotic agent, an antiviral agent, an antineoplastic agent, an immunotherapeutic agent, or a radiotherapeutic agent.

The phrase “anti-proliferative agent” can include agents that exert antineoplastic, chemotherapeutic, antiviral, antimitotic, antitumorigenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophyllotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

In some embodiments, a compound including or linked to a detectable moiety can be used in a method to detect and/or determine the presence, location, and/or distribution of cancer cells, i.e., cancer cells associated with RPTP cell adhesion molecules, in an organ or body area of a patient, e.g., at least one region of interest (ROI) of the subject. The ROI can include a particular area or portion of the subject and, in some instances, two or more areas or portions throughout the entire subject. The ROI can include regions to be imaged for both diagnostic and therapeutic purposes. The ROI is typically internal; however, it will be appreciated that the ROI may additionally or alternatively be external.

The presence, location, and/or distribution of the compound in the animal's tissue, e.g., brain tissue, can be visualized (e.g., with an in vivo imaging modality described above). “Distribution” as used herein is the spatial property of being scattered about over an area or volume. In this case, “the distribution of cancer cells” is the spatial property of cancer cells being scattered about over an area or volume included in the animal's tissue, e.g., brain tissue. The distribution of the agent may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject. It will be appreciated that the imaging modality may be used to generate a baseline image prior to administration of the composition. In this case, the baseline and post-administration images can be compared to ascertain the presence, absence, and/or extent of a particular disease or condition.

In one aspect, the compound including the detectable moiety may be administered to a subject to assess the distribution of cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of brain on the tumor margin that are outside the tumor edge that appear to be grossly normal but are infiltrated by dispersing tumor cells upon histological examination. For example, in glioma (brain tumor) surgery, the compound can be given intravenously about 24 hours prior to pre-surgical stereotactic localization MRI. The compounds can be imaged on gradient echo MRI sequences as a contrast agent that localizes with the glioma.

Compounds described herein that include a detectable moiety and specifically bind to and/or complex with RPTP cell adhesion molecules (e.g., PTPμ) expressed by cells or cancer cells can be used in intra-operative imaging (IOI) techniques to guide surgical resection and eliminate the “educated guess” of the location of the tumor margin by the surgeon. Previous studies have determined that more extensive surgical resection improves patient survival Stummer W, Novotny A, Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Fluorescence-guided resection of glioblastoma multiforme by using 5-aminolevulinic acid-induced porphyrins: a prospective study in 52 consecutive patients. Stummer W, Novotny A, Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Thus, compounds that function as diagnostic imaging agents have the potential to increase patient survival rates.

In some embodiments, to identify and facilitate removal of cancer cells, microscopic intra-operative imaging (IOI) techniques can be combined with systemically administered or locally administered compounds described herein. The compounds upon administration to the subject can target and detect and/or determine the presence, location, and/or distribution of cancer cells, i.e., cancer cells expressing RPTP cell adhesion molecules, in an organ or body area of a patient. In one example, the compound can be combined with IOI to identify malignant cells that have infiltrated and/or are beginning to infiltrate at a tumor brain margin. The method can be performed in real-time during brain or other surgery. The method can include local or systemic application of the compound described herein that includes a detectable moiety, e.g., a fluorescent or MRI contrast moiety. An imaging modality can then be used to detect and subsequently gather image data. The imaging modality can include one or combination of known imaging techniques capable of visualizing the compound. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

In one example, an agent including a compound linked to a fluorescent detectable moiety can be topically applied as needed during surgery to interactively guide a surgeon and/or surgical instrument to remaining abnormal cells. The compound may be applied locally in low concentration, making it unlikely that pharmacologically relevant concentrations are reached. In one example, excess material may be removed (e.g., washed off) after a period of time (e.g., incubation period).

In certain embodiments, the methods and compounds described herein can be used to measure the efficacy of a therapeutic administered to a subject for treating a metastatic, invasive, or dispersed cancer. In this embodiment, the compound can be administered to the subject prior to, during, or post administration of the therapeutic regimen and the distribution of cancer cells can be imaged to determine the efficacy of the therapeutic regimen. In one example, the therapeutic regimen can include a surgical resection of the metastatic cancer and the compound can be used to define the distribution of the metastatic cancer pre-operative and post-operative to determine the efficacy of the surgical resection. Optionally, the methods and compounds can be used in an intra-operative surgical procedure as describe above, such as a surgical tumor resection, to more readily define and/or image the cancer cell mass or volume during the surgery.

The compounds described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed compounds can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. In specific embodiments, oral, parenteral, or systemic administration are preferred modes of administration for treatment.

The compounds described herein can be administered alone as a monotherapy, or in conjunction with or in combination with one or more additional therapeutic agents. For example, the compounds described herein can be administered to the subject prior to, during, or post administration of an additional therapeutic agent and the distribution of metastatic cells can be targeted with the therapeutic agent. The agent can be administered to the animal as part of a pharmaceutical composition comprising the agent and a pharmaceutically acceptable carrier or excipient and, optionally, one or more additional therapeutic agents. The compound described herein and additional therapeutic agent can be components of separate pharmaceutical compositions, which can be mixed together prior to administration or administered separately. The compounds described herein, for example, be administered in a composition containing the additional therapeutic agent, and thereby, administered contemporaneously with the agent. Alternatively, the compounds described herein can be administered contemporaneously, without mixing (e.g., by delivery of the agent on the intravenous line by which the therapeutic agent is also administered, or vice versa). In another embodiment, the compounds described herein can be administered separately (e.g., not admixed), but within a short time frame (e.g., within 24 hours) of administration of the therapeutic agent.

The methods described herein contemplate single as well as multiple administrations, given either simultaneously or over an extended period of time. The compounds described herein (or composition containing the compounds) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the compounds and/or an additional therapeutic agent is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the compound in the subject, the agent can be administered between, for example, once a day or once a week.

For example, the administration of the compound and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

The compounds described herein and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of the compound described herein and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the compound described herein can also depend on the disease state or condition being treated along with the clinical factors and the route of administration of the compound.

The following example is included to demonstrate preferred embodiments.

### EXAMPLE

In this Example, we used a modelling approach, based on the structure of PTPσ, to identify a unique potential drug-binding pocket that sits at the interface between the D1 and D2 domains of PTPμ. We then used an artificial intelligence neural network (AtomNet® model) to identify small molecules predicted to interact with this region. The 75 best, chemically diverse, computational hits were tested for the ability to block glioma cell migration and growth as spheres in 3D culture. Active compounds were also tested for their ability to affect PTPμ-dependent adhesion likely through conformational effects to demonstrate an interaction with the intended target. We identified two high-priority compounds that inhibited glioma cell migration, sphere formation/growth and PTPμ-dependent adhesion. We also identified one activator of PTPμ-dependent adhesion and cancer cell migration. We propose that the high-priority inhibitors represent unique PTPμ-targeting agents that can be further developed to treat cancer and that the priority activator may inform how the D2 domain functions to regulate PTPμ.

### Methods

### Cell Culture

LN229 (LN-229), U87 (U87 MG) and Sf9 cells were purchased from the ATCC. The Gli36 (Gli36δ5) glioma cell line was provided by E. Chiocca and authenticated using IDEXX BioResearch (formerly RADIL: Research Animal Diagnostic Laboratory at the University of Missouri). The human glioma cell lines were cultured in DMEM (high glucose DMEM, Gibco)+10% (Gli36 and U87) or 5% (LN229) FBS (HyClone) at 37° C. and 5% CO₂. The Sf9 insect cells were cultured in Grace's complete medium (Gibco)+10% FBS at 27° C.

### Scratch-Wound Assays

Glioma cells (2.7×10⁴ cells per well) were seeded into Incuyte® Imagelock 96-well plates (Essen BioScience Inc.). Cells were incubated overnight to form monolayers; then, an IncuCyte® 96-well Wound-maker Tool was used, per the manufacturer's instructions, to generate uniform scratch wounds. Wound closure was observed using the IncuCyte live-cell imaging system as previously described.

### Glioma Sphere Assays

Ninety-six-well U-bottom plates were coated with 0.75% (wt/vol) PVA as previously described to create a non-adherent surface. A Leica CTR6500 microscope fitted with an automated stage was used to capture images on Day 1 and Day 7, and sphere footprint areas were measured using Image J (v1.52a, http://imagej.nih.gov/ij) as previously described. The effects of the compounds on cell condensation were quantified by calculating the normalized Day 1 footprint areas of the treated wells. Sphere growth was quantified by measuring the change in sphere footprint area (Day 1/Day 7×100) and normalizing that to the average size change of the matched DMSO controls. All values are presented as average percentages ±s.e.m.

### Helix Blue Staining

To test for non-specific toxicity, parental S9 cells (which lack PTPμ) were seeded into 96-well flat-bottomed tissue culture plates and treated with compounds (100 μM) or DMSO (1%) for 24 h. To test for glioma cell killing (which could be PTPμ-dependent), LN229 cells, seeded onto non-adherent surfaces as described above, were cultured with compounds (100 μM) for 24 h. Sf9 cells and LN229 Day 1 spheres were then stained with 5.5 μM helix blue and imaged im-mediately (Biolegend) on a Leica CTR6500 fluorescence microscope.

### Protein Tyrosine Phosphatase Mu-Dependent Aggregation Assay

Sf9 cells infected with baculovirus coding for human full-length PTPμ aggregate following rotation under low shear conditions. This procedure was adapted to a 48-well plate format for moderate-throughput drug screening as described.

### Results

### Identification a Potential Drug-Binding Pocket at the Interface Between the D1 and D2 Domains of Protein Tyrosine Phosphatase Mu

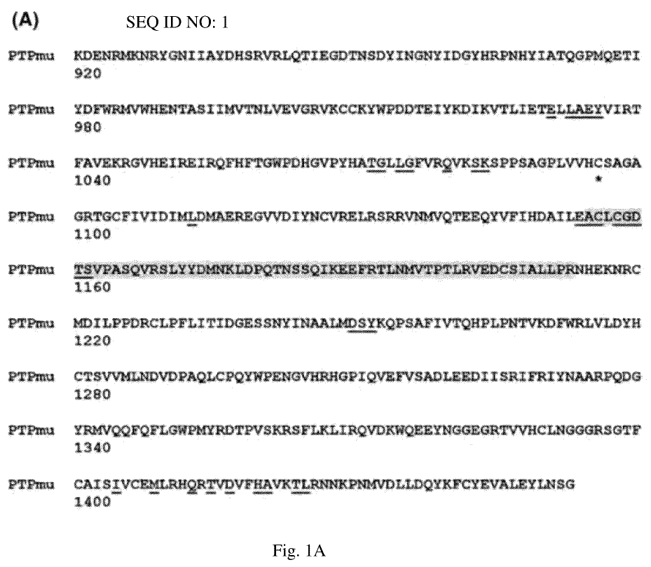

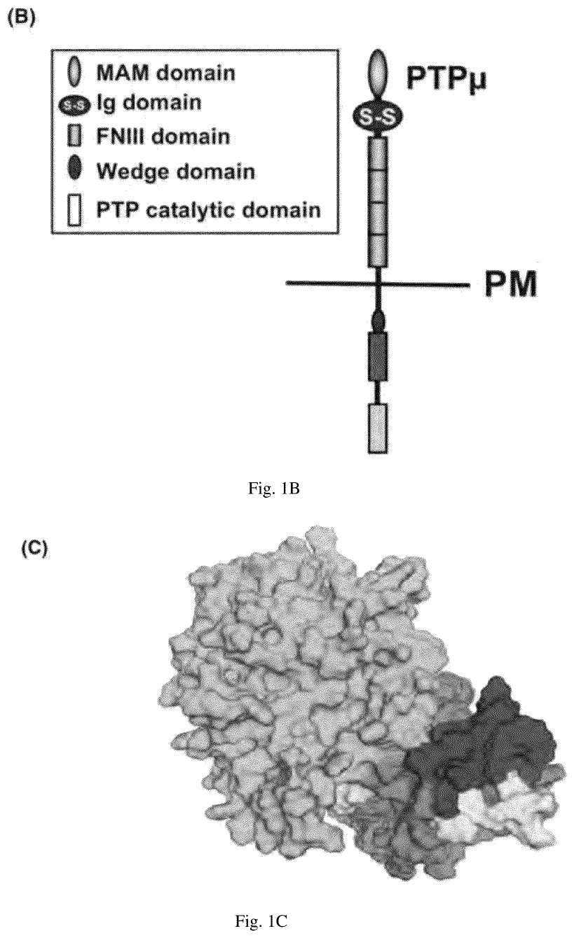

To date, all X-ray structures of RPTPs that contain both D1 and D2 domains show a similar orientation between the two domains, even though the linker region between them may allow for some flexibility in their relative alignment. Prompted by this (and in the absence of a crystal structure that includes the D2 domain of PTPμ), we modelled the D2 domain and parts of the N-terminal linker domain after the crystal structure for the related PTPσ [PDB ID 2FH7] and combined this with the available PTPμ structure for D1 [PDB ID 1 RPM] using ICM (v3.8-7 Molsoft L.L.C.) as previously described (FIG. 1). Our modelling efforts in a prior study focused on amino acids surrounding a binding pocket within the D1 domain, while this study focused on surface amino acids comprising an even deeper pocket between the D1 and D2 domains. In both cases, the surrounding amino acids showed strain energies of less than 7 kcal/mol. No attempts were made to resolve every energy strain in regions further away from our binding sites, especially in some of the flexible loop regions, as they were not relevant to the performance of our AtomNet predictions.

Existing crystal structures indicate a close association between the D1 and D2 domains and potential regulatory mechanisms based on dimerization. The crystal structure of the D1 domain of PTPα suggested the protein may exist as an autoinhibited homodimer in which the wedge domain of one molecule occludes the active site of the other. However, this auto-inhibited homodimer is incompatible with a complete D1-D2 structure and suggested a ‘head-to-toe’ autoinhibition model for PTPγ, where Asp residues (D1305, D1306) located on the loop connecting the β sheet strands β10 and β11 of the D2 domain, block access to the catalytic site on D1. PTPμ has just one Asp in the corresponding location (D1338, G1339), but in our modelled structure for PTPμ other potential interactions such as D1-wedge to D2 catalytic site-homologous region dimers are also generally possible (i.e., they do not lead to steric clashes between the domains). Clearly, a detailed investigation with for example protein-protein docking is necessary to assess the compatibility of the potential interaction sites. We hypothesized that targeting the potential interface between the D1 and D2 domains could lead to the discovery of inhibitors that function by locking the protein in an enzymatically inhibited conformation.

We previously identified a D1 wedge-adjacent pocket with drug-binding/functional activity so we took a similar approach with the D2 domain. We used the ICM pocket finder module (v3.8-7, Mol-soft L.L.C.) to identify a well-defined pocket at the interface between the D2 and the D1 domains and used the AtomNet® virtual screening platform to identify candidate D2-binding compounds. Atomwise performed a virtual high-throughput screen of 4 million compounds from the Mcule small-molecule library (version v20171018, https://mcule.com/) using their proprietary AI screening AtomNet® plat-form as described previously. Filtering of the 2000 top-scoring compounds via the removal of compounds with undesired chemical moieties followed by ECFP4 fingerprint-based Butina clustering (Tanimoto coefficient of 0.4 for similarity cut-off) provided a final selection of 75 chemically diverse compounds.

### Overview of the Functional Screen for Compounds Targeting the Protein Tyrosine Phosphatase Mu D2 Binding Pocket

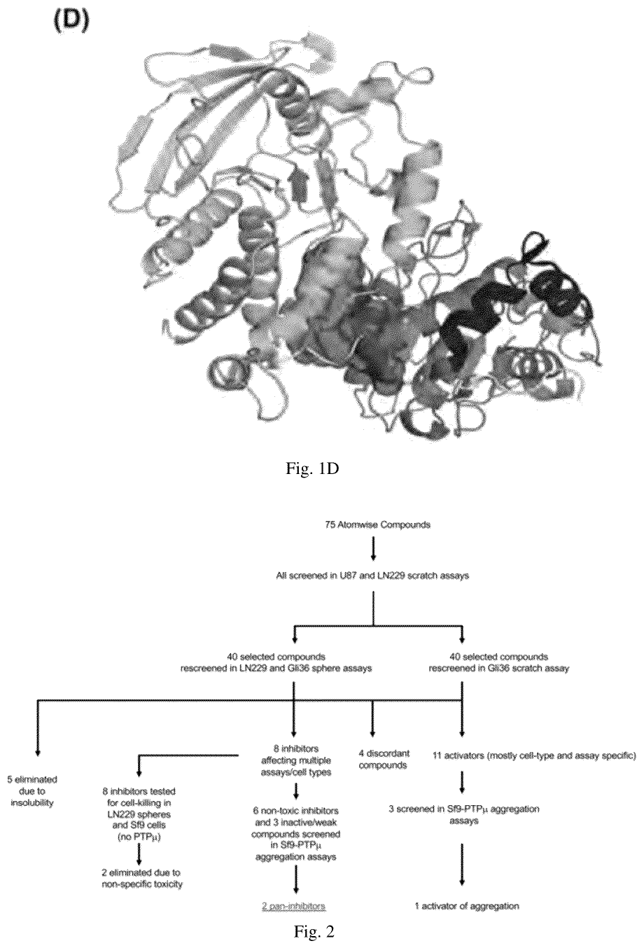

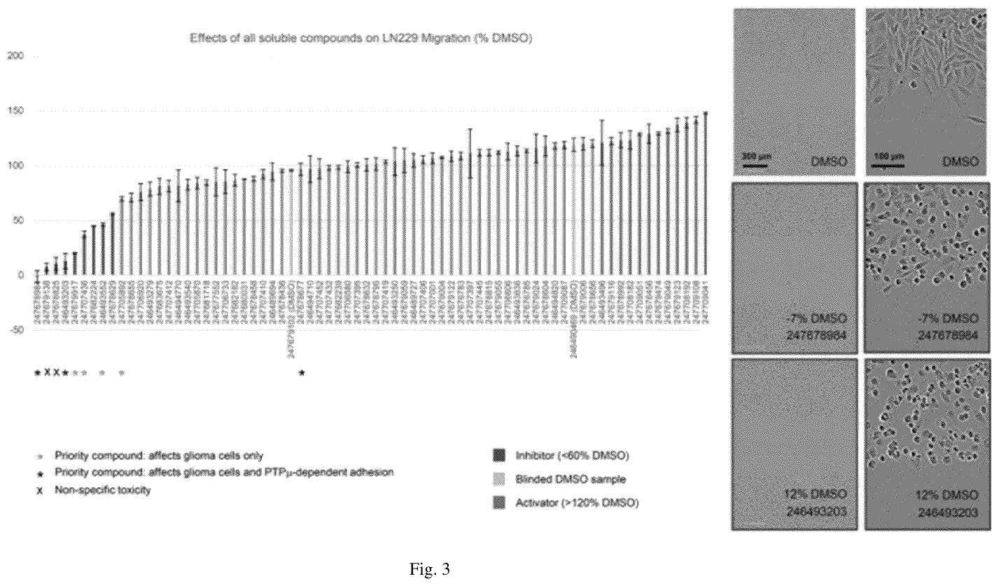

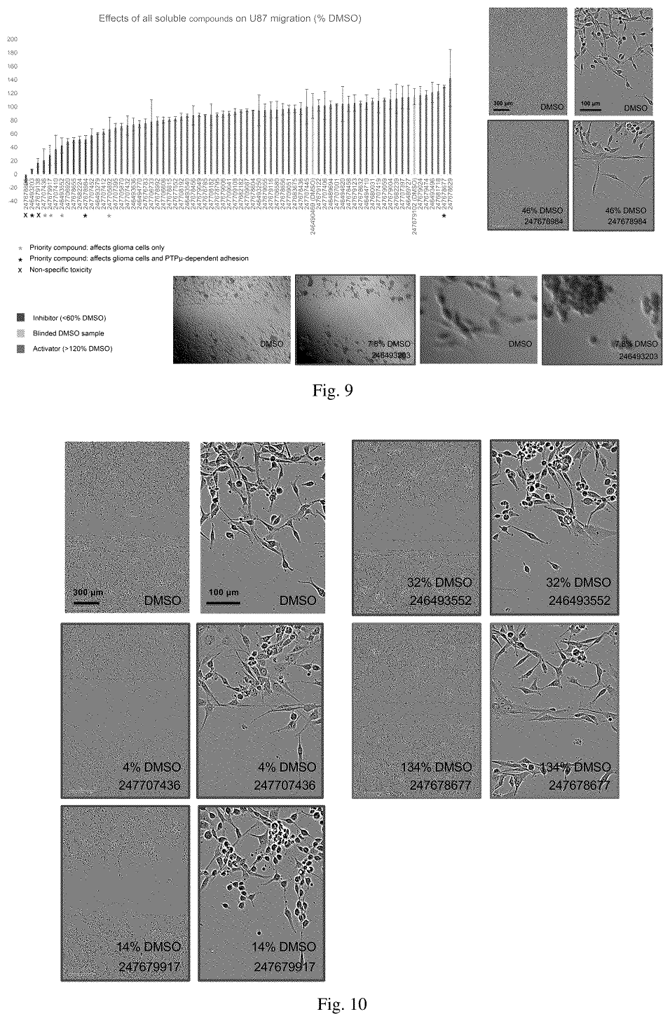

FIG. 2 shows a flowchart of our screening process. Atomwise provided 75 compounds predicted to interact with the D2 binding pocket (D2BP) and two-blinded DMSO controls. These were screened (at 100 μM) for effects on the migration of two glioma cell lines (LN229, FIG. 3 and FIG. 8 and U87 FIGS. 9 and 10) in scratch-wound assays. These two lines were chosen for the initial screen because they exhibit varying levels of fragment versus full-length PTPμ. LN229 cells express mostly PTPμ fragments and are invasive in orthotopic tumor models, whereas, U87 cells retain some full-length PTPμ and are non-invasive in vivo. A non-blinded vehicle control (DMSO) was used for normalization purposes in all assays.

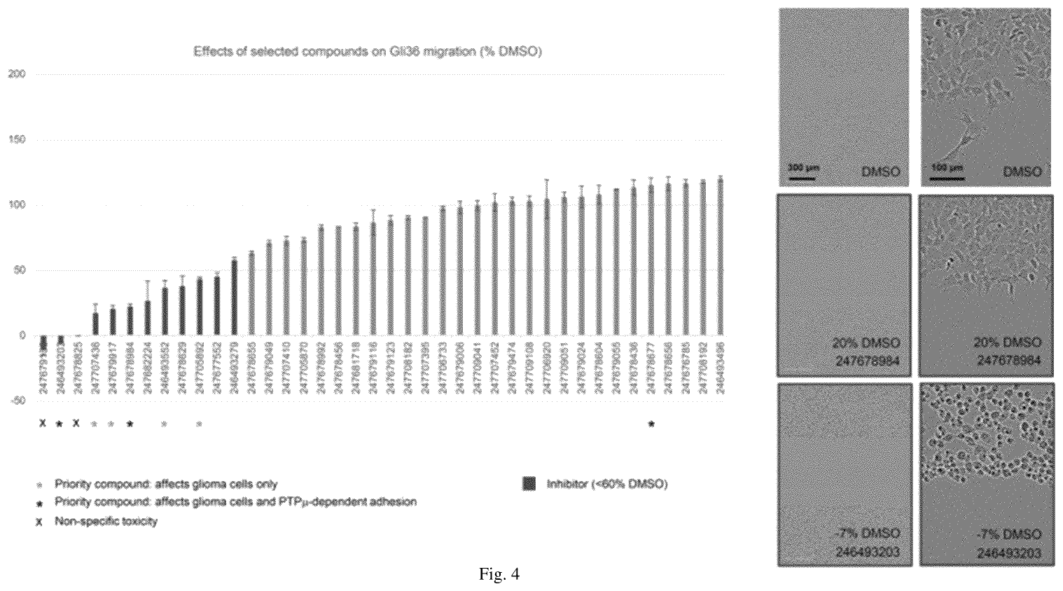

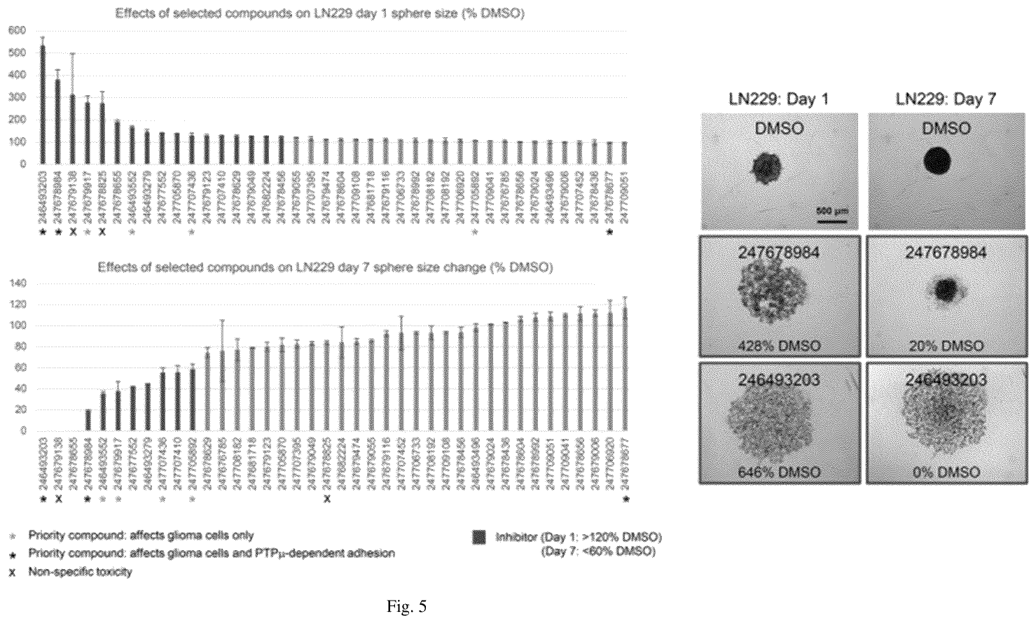

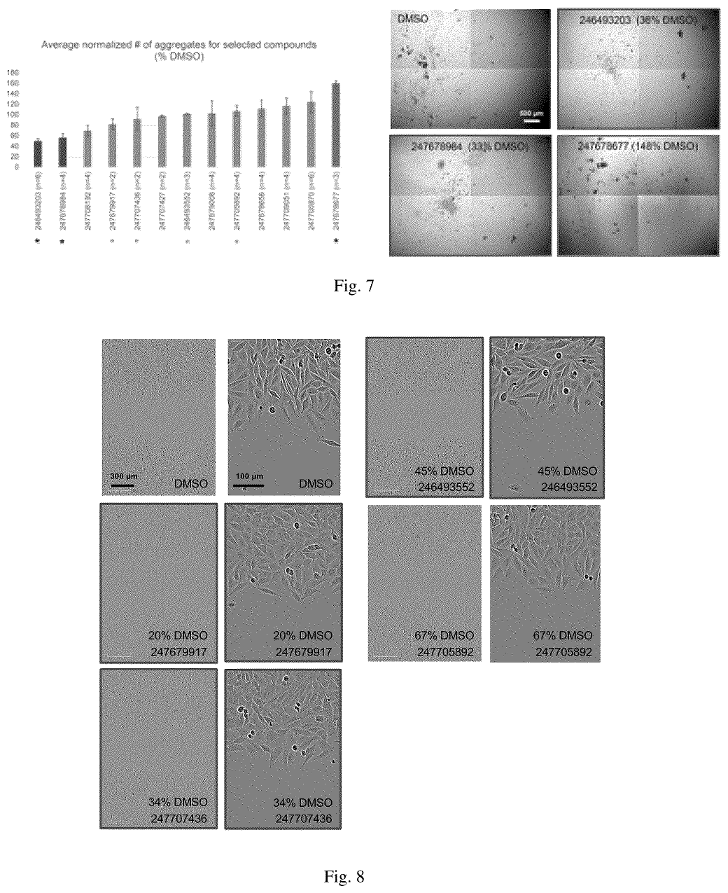

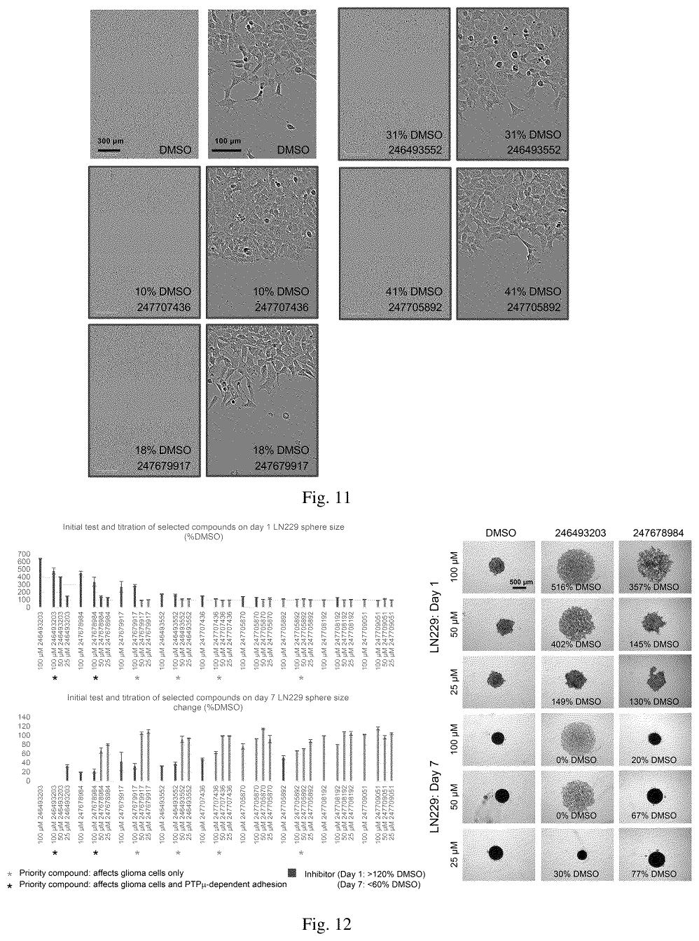

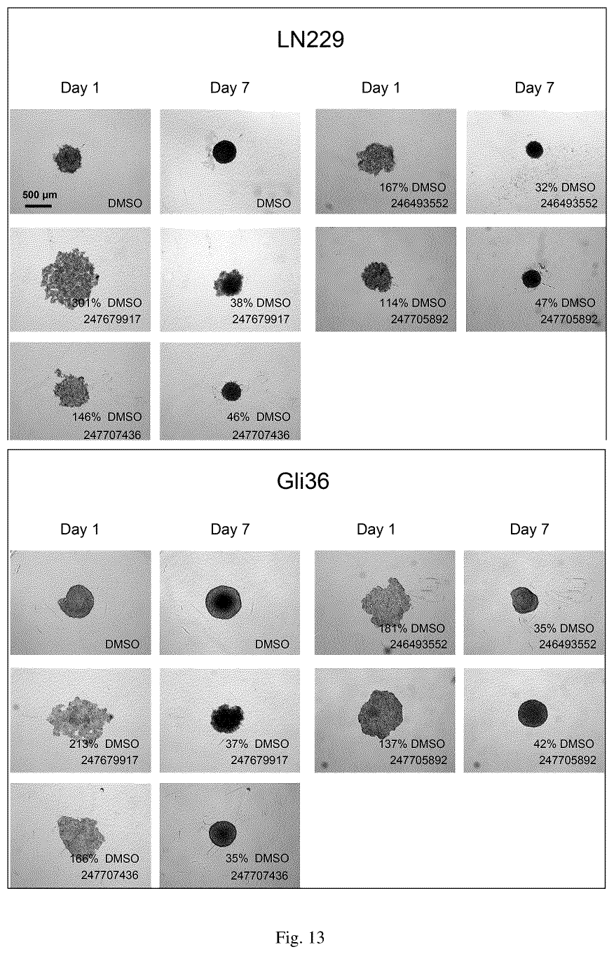

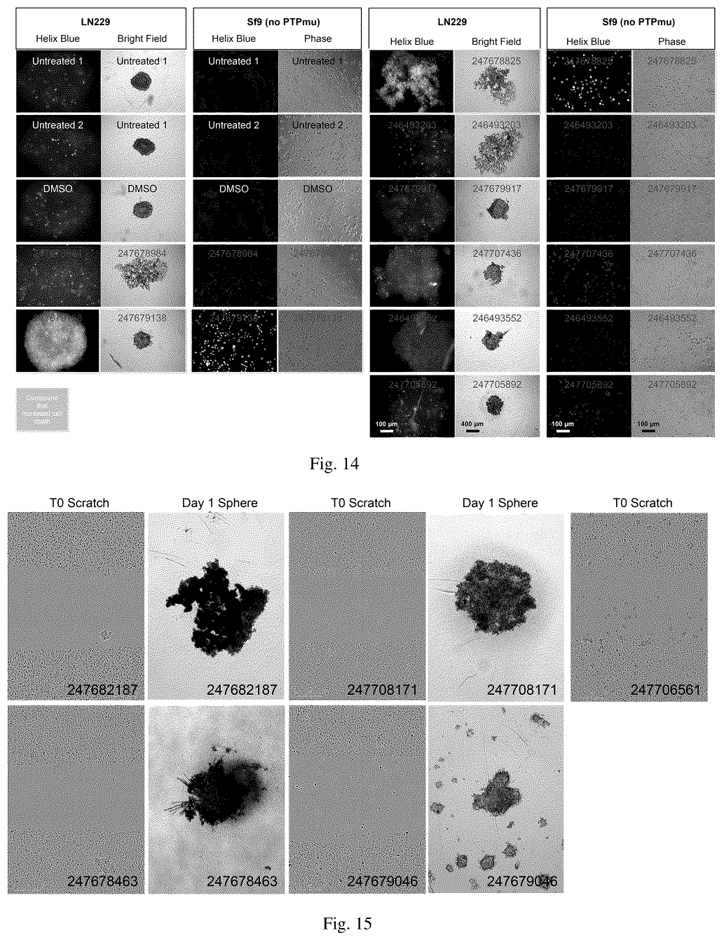

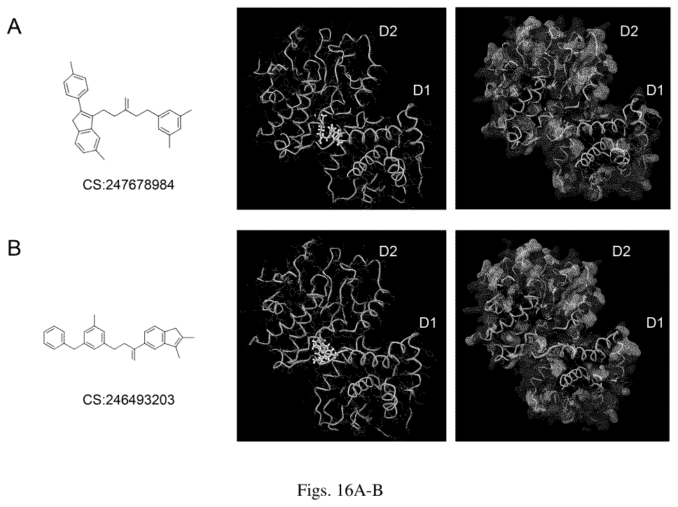

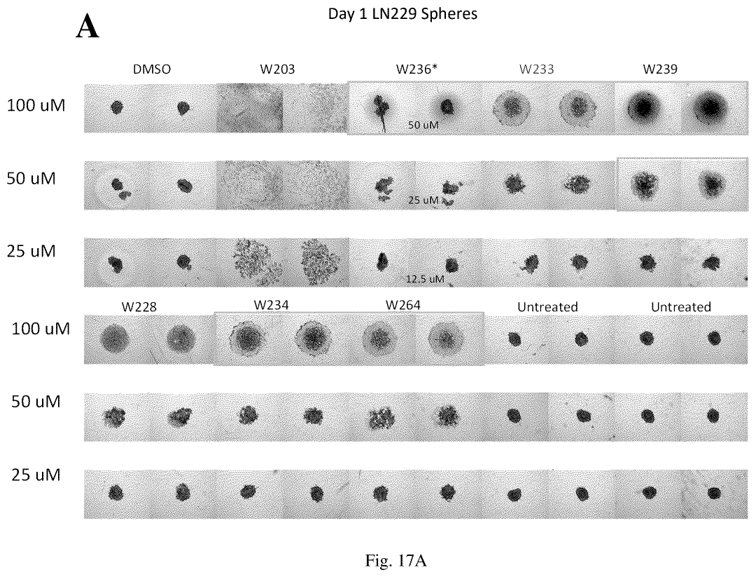

Forty compounds (13 inhibitors of LN229 and/or U87, 14 activators of LN229 and/or U87, one discordant compound [activated U87 and inhibited LN229] and 12 weak or apparently inactive compounds) were further tested for their ability to affect migration of a third glioma cell line (Gli36, FIG. 4 and FIG. 11) and the aggregation and growth of glioma cells in 3D culture (FIG. 5, 6 and FIGS. 12 and 13). Compounds exhibiting non-specific toxicity (n=2, FIG. 14) or insolubility (n=5, FIG. 15; which was typically more apparent in the sphere assay due to the U-bottom shape of the wells) were eliminated from consideration, and four discordant compounds were not considered further. Finally, a subset of glioma cell inhibitors and activators were tested for their ability to perturb PTPμ-dependent aggregation of Sf9 cells (FIG. 7). Parental Sf9 cells lack PTPμ, as well as other RPTPIIb subtypes and do not aggregate. However, they can be induced to aggregate by baculoviral-mediated overexpression of PTPμ, creating a highly specific assay for PTPμ-dependent adhesion. We found that two inhibitors that blocked the migration and sphere formation of every tested glioma cell type were also able to block Sf9-PTPμ aggregation. Additionally, one activator of U87 migration was able to stimulate Sf9 aggregation. Considering the D2 domain is primarily thought to have a regulatory role in controlling intra- or inter-molecular interactions that regulate PTPμ signaling, we had no preformed expectation of identifying D2BP compounds also able to alter aggregation. However, impinging on the D2 domain could change the architecture of the protein in a way that affects presentation of the extracellular segment, and because the Sf9 assay is highly specific for PTPμ, compounds (two inhibitors and one activator) that affect this assay are our highest priority hits and are indicated by black asterisks (FIGS. 3-7, FIGS. 9 and 12). The carbon skeletons of the two high-priority inhibitors along with docking models and estimated Gibb's free energies of binding are presented in FIG. 16. The four compounds that inhibited glioma cells but not PTPμ-mediated aggregation may still have therapeutic potential and are marked by orange asterisks. They may alter PTPμ's signaling function without altering adhesion or act via another mechanism.

### Glioma Cell Scratch Assays

Cancer cell motility was evaluated by scratch assays where cells migrate into a wound and close it creating a monolayer. The effects of all soluble D2BP compounds on LN229 scratch wound closure are shown in FIG. 3. In this initial screen, nine strong LN229 inhibitors were identified (red bars), and of these, six were eventually prioritized for being effective in multiple cell types and assays (FIG. 2). We also identified 12 activators (≥120% of DMSO closure rate) of LN229 wound closure, only one of which (246493496) was shared with U87 cells (FIG. 9). In general, the U87 cells appeared less ‘activatable’, with only four compounds stimulating their migration by ˜1.2-1.4×, one of which (247678677) was later shown to affect PTPμ-dependent adhesion. Thus, 247678677 is indicated as a priority compound in FIG. 2 despite not affecting LN229 migration. It is possible that this compound was a U87-specific activator because these cells express more full-length PTPμ than LN229 cells.

FIG. 3 shows examples of the morphology of LN229 cells that were treated with DMSO (1%) or two high-priority inhibitors (100 μM). Within DMSO-treated monolayers, the LN229 cells were generally spindle shaped, whereas cells at the wound edge were flattened and showed lamellipodial ruffles consistent with being migratory. The two high-priority inhibitors caused LN229 cells to round up. 246493203 caused similar morphological changes in U87 cells, but 247678984 had a more modest effect on this cell type, and there was little difference in morphology between U87 cells treated with this compound and control cells (FIG. 9).

The morphological effects of the low-priority inhibitors on LN229 and U87 cells are shown in FIGS. 8 and 10, respectively, and were generally quite subtle. LN229 cells within monolayers treated with 247679917, 247707436 and 247705892 appeared more spread than spindle shaped, creating a cobblestone appearance, and cells at the edges of the scratches exhibited few ruffles consistent with reduced motility. 246493552 reduced the appearance of ruffles at the scratch edge but did not affect the overall appearance of the monolayer. Some rounding of U87 cells was ap-parent with 247679917 and 246493552, but 247707436, despite being strongly inhibitory, did not affect the morphology of this cell type. The U87-specific priority activator did not produce obvious morphological changes.

A selection of 40 compounds (those shown to inhibit or activate the migration of one or both cell types and some inert control compounds) were tested for effects on Gli36 cells (FIG. 4). This line expresses mostly PTPμ fragments and migrates rapidly in vitro (requiring scratch-wound endpoint images to be taken at 8 h versus the typical 12-h time frame for the U87 and LN229 experiments). Despite this, the majority of rescreened inhibitors were effective on this cell line; however, no Gli36 activators were identified. Considering the rapid migration of Gli36 cells, they may not be particularly sensitive to further stimulation.

Representative endpoint images of Gli36 scratch wounds treated with DMSO or the two high-priority inhibitors are shown in FIG. 4. Gli36 cells in control monolayers have a cobblestone appearance, while those at the scratch edge appear to extend process and move into the scratch as linked chains of cells. The strong priority inhibitor 246493203 caused cell rounding while the more moderate inhibitor 247678984 reduced the appearance of cell chains moving into the scratch. The effects of the low-priority inhibitors are shown in FIG. 11. Of these, only 247707436 produced a marked change in cell morphology. It completely blocked the formation of processes and cell chains at the edge of the scratch.

### Glioma Cell Sphere Assays

Glioma cells cultured on non-adherent surfaces will self-adhere and grow in culture as a compact sphere that mimics some aspects of the 3D tumor microenvironment. Agents able to affect the growth and survival of 3D cultures may be more potent in vivo anti-tumor agents than those just able to affect cells in 2D culture. Also, culture systems that favor cell-cell over cell-substrate adhesion are likely better models for understanding the role of cell-cell adhesion molecules in cancer biology. Thus, the effects of 40 compounds (13 shown to inhibit the migration of at least one cell type, 14 shown to stimulate migration of at least one cell type, one discordant com-pound [activated U87 and inhibited LN229] and 12 weak or apparently inactive compounds) were tested for their ability to affect LN229 sphere formation and growth (FIG. 5). To quantify sphere formation, the footprint areas occupied by the cells were measured on Day 1, and the values normalized to the average footprint area of the DMSO controls. Larger footprint sizes (>120%) on Day 1 indicate inhibition, i.e., the cells have failed to compact. Sphere growth was quantified by measuring the percent changes in sphere footprint areas between Day 1 and Day 7 and normalizing those to the average growth of the DMSO controls. On Day 7, spheres exhibiting a size change ≤60% of controls were considered inhibited. Samples that fell apart on Day 1 or during the culture period are displayed as having 0% growth.

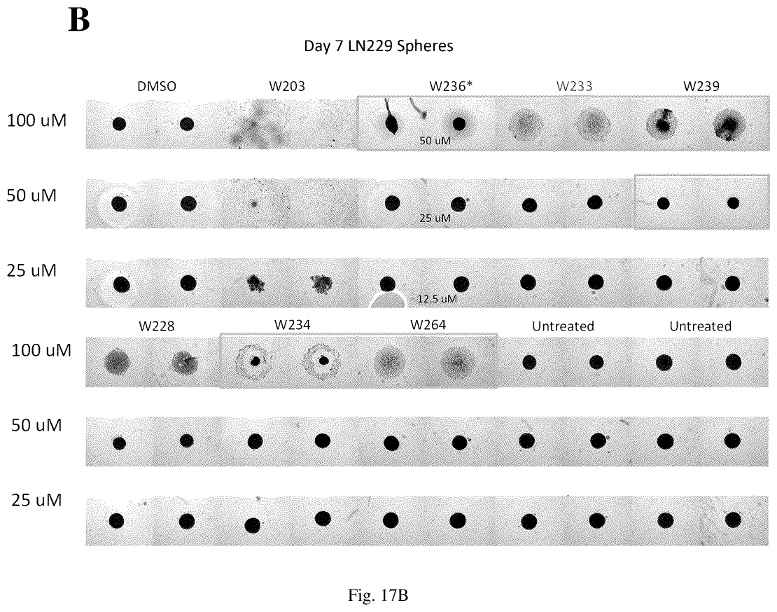

Representative images of LN229 spheres cultured in the presence of DMSO or the high-priority inhibitors are shown in FIG. 5. The LN229 cells treated with DMSO formed a loose aggregate on Day 1, and by Day 7, this aggregate had formed a compact sphere. In contrast, the cells treated with a strong inhibitor (246493203) failed to compact and remained as single cells at the bottom of the well. Samples treated with the more moderate inhibitor (247678984) formed scattered clumps on Day 1 that eventually compacted into a small aggregate by Day 7. All prioritized inhibitors were titrated to estimate a minimal dose (25, 50 or 100 μM) able to affect sphere formation and/or growth (FIG. 12 and FIG. 17). The strong inhibitor 246493203 dramatically disrupted sphere formation at 100 μM and 50 μM. At 25 μM, sphere formation was modestly perturbed, but the resulting aggregates grew poorly in culture. The other high-priority inhibitor (247678984) was most effective at 100 μM where it slowed both sphere condensation and growth. At lower doses, that compound still had a modest effect on sphere formation, but the samples recovered and grew normally. The low-priority inhibitors (orange asterisks) were only effective at 100 μM, and representative images of samples treated with these compounds are shown in FIG. 13. Three of these compounds (247707436, 246493552 and 247705892) produced very similar effects: sphere formation was modestly slowed, and the resulting aggregates grew poorly in culture. 247679917 caused a distinct effect. Cells treated with this compound aggregated poorly but eventually condensed into a clump with irregular loose-appearing margins.

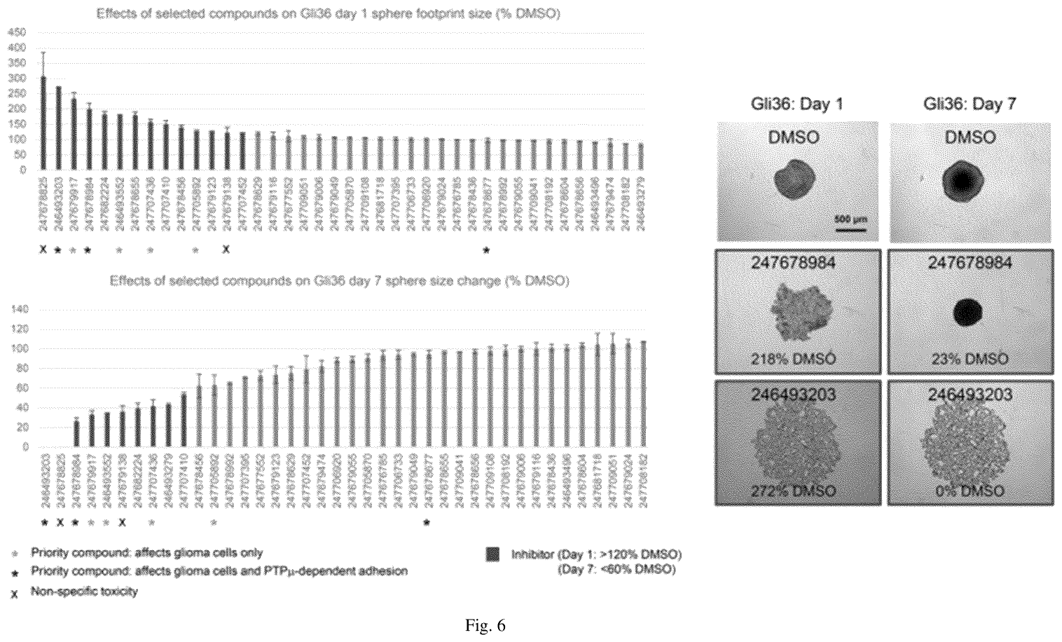

The effects of the priority inhibitors on Gli36 sphere formation and growth were generally similar (i.e., treatment with the compounds slowed condensation and/or growth; FIG. 6 and FIG. 13). 247679917 was again distinct. Like LN229 cells, Gli36 cells responded to this compound by forming loose-appearing clumps with irregular borders.

### Protein Tyrosine Phosphatase Mu-Dependent Aggregation Assays

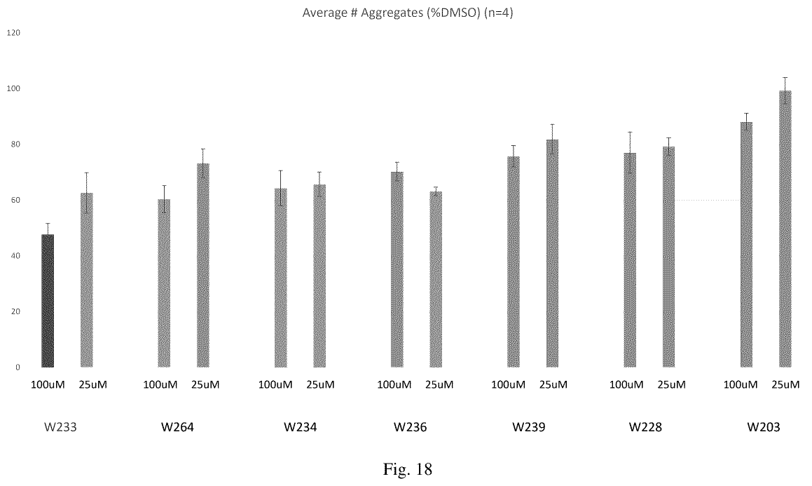

The glioma cell assays described above involve hours (scratch assays) to many days (sphere assays) of treatment and a very high dose of compounds, raising the possibility of off-target effects. To maximize our chances of identifying PTPμ-targeting compounds, we screened selected hits in a short-term assay for PTPμ-dependent adhesion. RPTPIIb family members are not expressed in parental Sf9 cells, and these cells do not aggregate. However, they can be induced to rapidly aggregate in response to baculoviral-driven expression of PTPμ, providing a highly specific measure of PTPμ function. It is unclear how the D2 domain might impact adhesion, but it could participate in interactions that affect PTPμ oligomerization or association with downstream targets, thereby altering the PTPμ-adhesive complex. To test if our D2BP-targeted compounds affect adhesion, PTPμ-expressing Sf9 cells were treated with selected compounds (100 μM) for 20 min then induced to aggregate by rotation. Aggregates above an arbitrary threshold size (4000 μm²) were then counted and normalized to the number present in the DMSO-treated controls. End-point images of samples treated with DMSO, priority inhibitors and one priority activator are shown in FIG. 7 and FIG. 18. The DMSO-treated sample exhibits many variable-sized aggregates, whereas, the samples treated with the high-priority inhibitors (247678984 and 246493203) have fewer/smaller aggregates. In contrast, samples treated with the U87 activator 247678677 (FIG. 9) exhibit an increase in the number of intermediate-sized aggregates.

Through computational modelling, we have identified a pocket at the interface between the D1 and D2 domains of PTPμ that can be exploited for drug development. Compounds predicted to interact with this region were identified through an AI-based computational algorithm and tested for their ability to affect tumor cell migration, growth in 3D culture and PTPμ-dependent adhesion. We identified two compounds that inhibited multiple glioma cell types and reduced PTPμ-dependent adhesion, the strongest of which (246493203) was effective on glioma cell spheres down to 25 μM. We also identified four low-priority inhibitors that inhibited glioma cell migration and sphere formation/growth without affecting PTPμ-aggregation and one activator of PTPμ-dependent aggregation that also stimulated U87 migration. We propose that the high-priority inhibitors may serve as a starting point for developing PTPμ-specific therapeutic agents. The low-priority inhibitors may affect PTPμ-signaling without altering PTPμ-dependent adhesion or act via other mechanisms. They may still be therapeutically useful considering their ability to inhibit glioma cells in culture, but they were only active at very high concentrations, meaning they would require considerable refinement. The activator is also interesting, not from a therapeutic standpoint for cancer cells, but because it might inform how the D2 domain functions to regulate PTPμ. Notably, the results of this screen were very similar to those of a prior screen for compounds targeting a D1 wedge-adjacent binding pocket. In each screen, we recovered inhibitors able to affect both glioma cell migration and PTPμ-dependent adhesion and activators with the converse effect. These correlations led us to hypothesize that cancer cells may actually receive a growth/motility benefit from increased PTPμ-mediated adhesion. This is in contrast to the tumor suppressive function proposed for PTPμ and may indicate that glioma cells have subverted the normal adhesive role of full-length PTPμ (in contact inhibition) into adhesive signals, possibly mediated by shed fragments, that promote cancer progression.

We have identified small molecules predicted to interact with a unique regulatory site within the D2 domain of PTPμ that inhibit PTPμ-dependent adhesion, glioma cell migration, sphere formation and growth in 3D culture. These compounds may serve as the basis for developing new treatments for cancers where the function of PTPμ is dysregulated, including glioblastoma and gynecological cancers.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, the following is claimed:

1. A method of detecting, monitoring, and/or imaging cancer cells and/or cancer cell metastasis, migration, dispersal, and/or invasion, and/or for treating cancer in a subject, the method comprising:
administering to the subject a pharmaceutical composition comprising: a compound of formula (IA): 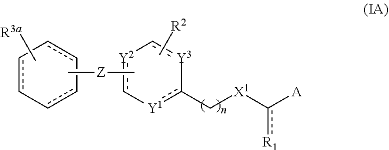
or a pharmaceutically acceptable salt, tautomer, or solvate thereof,
wherein:
a dashed line is an optional bond;
A is 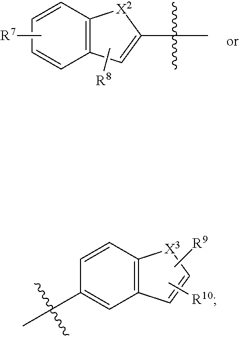
X¹ is CH₂ or O;
X² and X³ are each independently N(R¹³) or CH₂;
Y¹, Y², and Y³ are each independently C(H)ₘ or N(H)ₙ;
Z is an alkylene, -alkylene-cycloalkylene-, -alkylene-heterocyclylene-, or —N(R⁶)—, each optionally substituted with one or more R⁵,
R¹ and R⁵ are each independently O, —N(R⁶)₂, halogen, hydroxyl, alkyl, haloalkyl, or alkoxy;
R² and each R^{3a} are each independently absent, —N(R⁶)₂, halogen, hydroxyl, alkyl, haloalkyl, alkoxy;
R⁴ is ═O, halogen, hydroxyl, alkyl, alkoxy, or haloalkyl;
R⁶ is H, halogen, hydroxyl, alkyl, alkoxy, or haloalkyl;
R⁷, R⁸, R⁹, and R¹⁰ are each independently absent, halogen, hydroxyl, alkyl, alkoxy, or haloalkyl; and
R¹³ is H, halogen, hydroxyl, alkyl, alkoxy, or haloalkyl;
m is 0, 1, or 2; and
n and n¹ are each independently 0 or 1.

2. The method of claim 1, wherein X² is N(R¹³) or wherein X³ is N(R¹³).

3. The method of claim 1, wherein A is selected from: 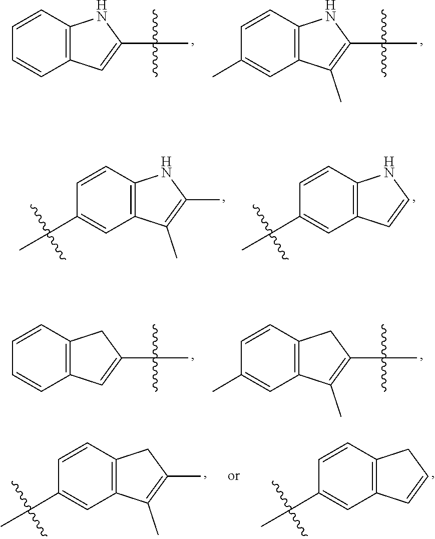
each of which is optionally substituted with a halogen.

4. The method of claim 1, wherein R¹ is O.

5. The method of claim 1, wherein X¹ is O.

6. The method of claim 1, wherein n¹ is 1 and Y¹, Y², and Y³ are each independently N; and wherein R² is —N(R⁶)₂.

7. The method of claim 1, wherein is n¹ is 0 and Y¹, Y², and Y³ are each independently CH; and wherein R² is absent.

8. The method of claim 1, wherein Z is: 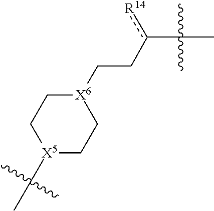
or —N(H)—; X⁵ and X⁶ are each independently CH or N; and R¹⁴ is O, —N(H)₂, halogen, hydroxyl, alkyl, haloalkyl, or alkoxy.

9. The method of claim 1, wherein Z is 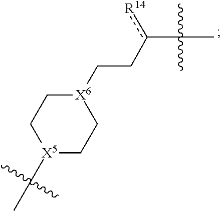
X⁵ and X⁶ are each independently CH or N; and R¹⁴ is O; or wherein Z is —N(H)—, and R^{3a} is absent.

10. The method of claim 1, wherein the compound of formula (IA) is a compound of formula (II): 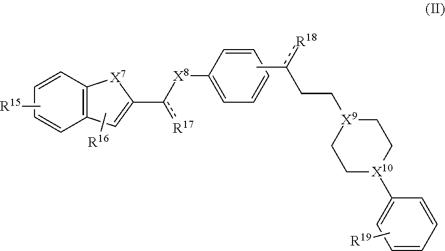
or a pharmaceutically acceptable salt, tautomer, or solvate thereof;
wherein,
a dashed line is an optional bond;
X⁷ is N(R²⁰) or CH₂;
X⁸ is CH₂ or O;
X⁹ and X¹⁰ are each independently CH or N;
R¹⁵ and R¹⁶ are each independently absent, halogen, hydroxyl, alkyl, alkoxy, or haloalkyl;
R¹⁷ and R¹⁸ are each independently O, —N(R²¹)₂, halogen, hydroxyl, alkyl, haloalkyl, or alkoxy;
R¹⁹ is absent, —N(R²⁰)₂, halogen, hydroxyl, alkyl, haloalkyl, alkoxy; and
each R²⁰ is independently H, halogen, hydroxyl, alkyl, alkoxy, or haloalkyl.

11. The method of claim 10, wherein X⁷ is N(R²⁰); X⁸ is O; X⁹ and X¹⁰ are N; R¹⁵ and R¹⁶ are each independently absent, halogen, hydroxyl, C₁-C₆ alkyl, C₁-C₆ alkoxy, or C₁-C₆ haloalkyl; R¹⁷ and R¹⁸ are each independently O; R¹⁹ is absent, halogen, or alkoxy; and R²⁰ is H, halogen, C1-C₆ alkyl, or C₁-C₆ haloalkyl.

12. The method of claim 1, wherein the compound or formula (IA) is a compound of formula (III): 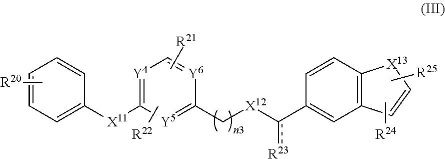
or a pharmaceutically acceptable salt, tautomer, or solvate thereof;
wherein:
a dashed line is an optional bond;
X¹¹ and X¹³ are each independently N(R²⁶) or CH₂;
X¹² is CH₂ or O;
Y⁴, Y⁵, and Y⁶ are each independently C(H)ₘ or N(H)ₙ
X⁸ is CH₂ or O;
X⁹ and X¹⁰ are each independently CH or N;
R²⁰, R²⁴ and R²⁵ are each independently absent, halogen, hydroxyl, alkyl, alkoxy, or haloalkyl;
R²¹ and R²² are each independently absent, —N(R²⁰)₂, halogen, hydroxyl, alkyl, haloalkyl, alkoxy;
R²³ is O, —N(R¹⁸)₂, halogen, hydroxyl, alkyl, haloalkyl, or alkoxy;
each R²⁶ is independently H, halogen, hydroxyl, alkyl, alkoxy, or haloalkyl; and
n³ is 0 or 1.

13. The method of claim 1, wherein the compound of formula (IA) is selected from: 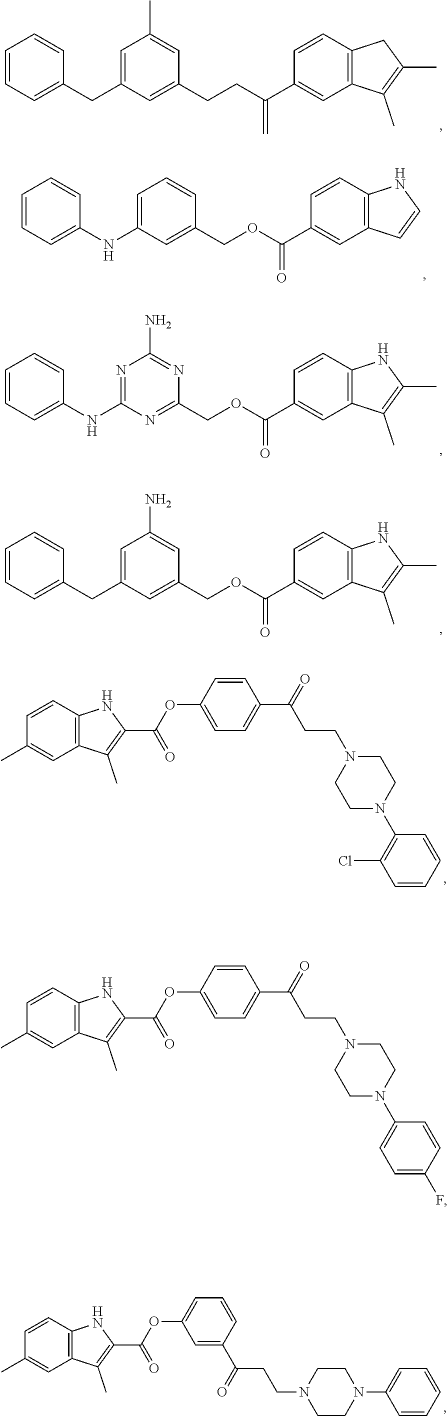 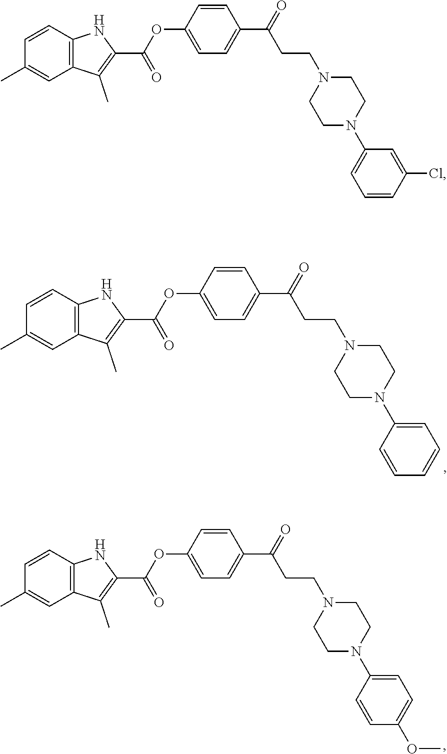
or a pharmaceutically acceptable salt, tautomer, or solvate thereof.

14. The method of claim 1, wherein the compound specifically binds to and/or complexes with an intracellular portion or fragment of an RPTP cell adhesion molecule that is expressed by a cancer cell or another cell in the cancer cell microenvironment.

15. The method of claim 1, wherein the compound further includes a detectable moiety linked to and/or complexed with the compound, the detectable moiety including at least one of a contrast agent, imaging agent, radiolabel, semiconductor particle, or nanoparticle.

16. The method of claim 1, wherein the compound inhibits glioma cell migration in a scratch wound healing assay at least about 5% compared to glioma cells administered DMSO.

17. The method of claim 1, wherein the compound inhibits aggregation of glioma sphere formation at least about 5% compared to glioma cells administered DMSO.

18. The method of claim 1, wherein the compound inhibits aggregation of PTPμ expressing SFF9 cells at least about 5% compared to PTPμ expressing SFF9 cells administered DMSO.

19. The method of claim 1, wherein the compound inhibits PTPμ's enzymatic activity in an in vitro phosphatase assay at least about 5% compared DMSO.

20. The method of claim 1, wherein the compound inhibits tumor growth at least about 5% compared DMSO.


SEQUENCE LISTING (1 sequence)

SEQ ID NO: 1 - protein, 533 aa, synthetic construct
KDENRMKNRYGNITAYDHSRVRLQTIEGDTNSDYINGNYIDGYHRPNHYIATQGPMQETIYDFWRMVWHENTASIIMVTNLVEVGRVKCCKYWPDDTEIYKDIKVTLIETELLAEYVIRTFAVEKRGVHEIREIRQFHFTGWPDHGVPYHATGLLGFVRQVKSKSPPSAGPLVVHCSAGAGRTGCFIVIDIMLDMAEREGVVDIYNCVRELRSRRVNMVQTEEQYVFIHDAILEACLCGDTSVPASQVRSLYYDMNKLDPQTNSSQIKEEFRTLNMVTPTLRVEDCSIALLPRNHEKNRCMDILPPDRCLPFLITIDGESSNYINAALMDSYKQPSAFIVTQHPLPNTVKDFWRLVLDYHCTSVVMLNDVDPAQLCPQYWPENGVHRHGPIQVEFVSADLEEDIISRIFRIYNAARPQDGYRMVQQFQFLGWPMYRDTPVSKRSFLKLIRQVDKWQEEYNGGEGRTVVHCLNGGGRSGTFCAISIVCEMLRHQRTVDVFHAVKTLRNNKPNMVDLLDQYKFCYEVALEYLNSG